(19) United States
(12) Patent Application Publication
(10) Pub. No.: US 2026/0317279 A1
(43) Pub. Date: Oct. 15, 2026

(54) SOLID STATE SALIFICATION OF ORGANIC MOLECULES

(71) Applicant: Florida State University Research Foundation, Inc., Tallahassee, FL (US)

(72) Inventors: Zachary T. Dowdell, Tallahassee, FL (US); Robert W. Schurko, Tallahassee, FL (US)

(21) Appl. No.: 19/712,248

(22) Filed: Jun. 18, 2026

Related U.S. Application Data
(63) Continuation-in-part of application No. 19/637,394, filed on Apr. 2, 2026.
(60) Provisional application No. 63/782,712, filed on Apr. 3, 2025.

(51) Int. Cl. C07D 213/803; C07C 227/44; C07C 231/22; C07C 277/08; C07C 303/42; C07D 213/81; C07D 213/82; C07D 213/86; C07D 233/64; C07D 473/08; C07D 473/10; C07D 473/12; C07D 499/14

(52) U.S. Cl. CPC C07D 213/803; C07C 227/44; C07C 231/22; C07C 277/08; C07C 303/42; C07D 213/81; C07D 213/82; C07D 213/86; C07D 233/64; C07D 473/08; C07D 473/10; C07D 473/12; C07D 499/14

(57) ABSTRACT

In accordance with the purpose(s) of the present disclosure, as embodied and broadly described herein, the disclosure, in one aspect, relates to methods for producing a salt of a solid organic compound. The method involves (a) combining in the solid state the solid organic compound comprising one or more protonatable groups and an acid composition, wherein the amount of acid in the acid composition is about stoichiometric or less than stoichiometric relative to the one or more protonatable groups of the solid organic compound to produce a precursor mixture; and mixing the precursor mixture by mechanochemical milling to produce the salt of the solid organic compound. The methods described herein can produce salts of a variety of different organic molecules and is capable of being scaled up.

### CROSS-REFERENCE TO RELATED APPLICATIONS

This application is a continuation-in-part of U.S. Nonprovisional patent application Ser. No. 19/637,394, filed on Apr. 2, 2026, which claims the benefit of and priority to U.S. Provisional Patent Application No. 63/782,712, filed on Apr. 3, 2025, the contents of which are incorporated by reference herein in their entireties.

### STATEMENT ON FUNDING PROVIDED BY THE U.S. GOVERNMENT

This invention was made with Government support under contract DMR2128556 awarded by the National Science Foundation. The Government has certain rights in the invention.

### BACKGROUND

Salts of organic molecules have widespread applications in several industries including pharmaceuticals, nutraceuticals, agrochemicals, and explosives. Typically, the free base of the organic compound is mixed with an excess amount of acid in a solvent to produce the corresponding salt. However, undesirable reactions such as acid hydrolysis can occur. Thus, it would be desirable to have alternative approaches to producing salts of organic compounds in a straight forward and efficient manner.

### SUMMARY

In accordance with the purpose(s) of the present disclosure, as embodied and broadly described herein, the disclosure, in one aspect, relates to methods for producing a salt of a solid organic compound. The method involves (a) combining in the solid state the solid organic compound comprising one or more protonatable groups and an acid composition, wherein the amount of acid in the acid composition is about stoichiometric or less than stoichiometric relative to the one or more protonatable groups of the solid organic compound to produce a precursor mixture; and mixing the precursor mixture by mechanochemical milling to produce the salt of the solid organic compound. The methods use acid compositions that have very little solvent (e.g., microliter scale relative to the amount of the organic molecule). The methods described herein can produce salts of a variety of different organic molecules and is capable of being scaled up.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows the synthetic pathways for the various solid forms of Picolinic Acid.

FIG. 2 shows the experimental and simulated PXRD patterns of nicotinamide and HCl salts of nicotinamide.

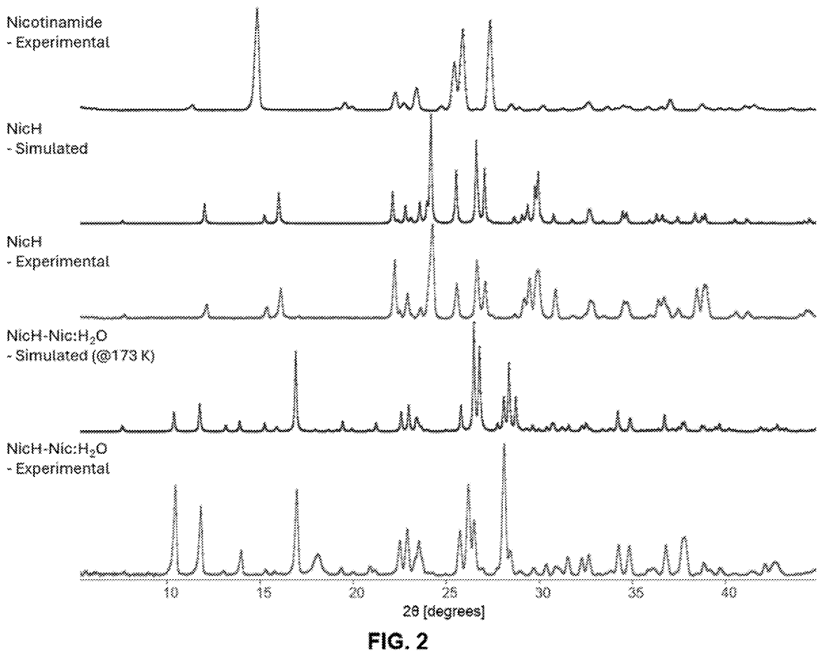

FIG. 3 shows the experimental and simulated PXRD patterns of nicotinamide and salts of nicotinamide.

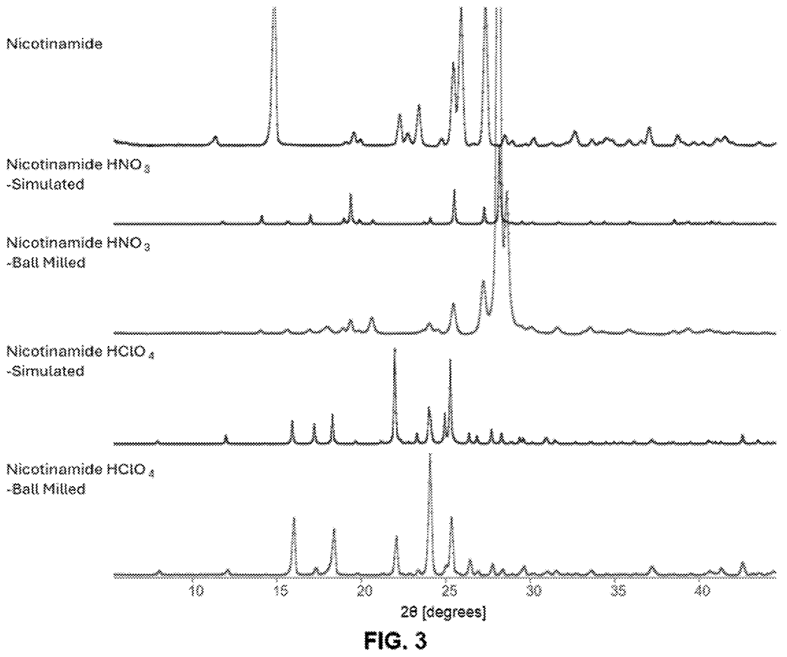

FIG. 4 shows the experimental and simulated PXRD patterns of isonicotinamide and salts of isonicotinamide.

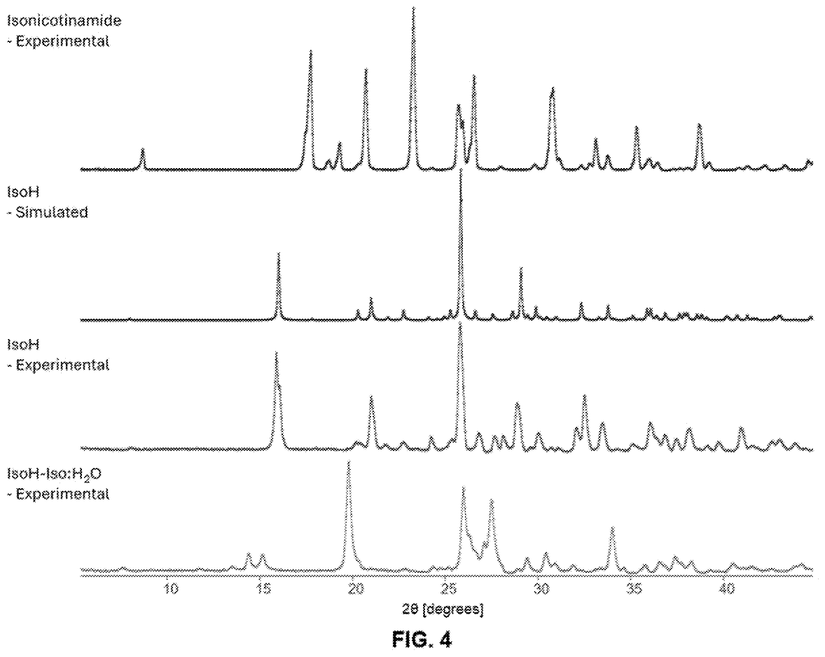

FIG. 5 shows the experimental and simulated PXRD patterns of picolinamide and salts of picolinamide.

FIG. 6 shows the experimental and simulated PXRD patterns of nicotinic acid and salts of nicotinic acid.

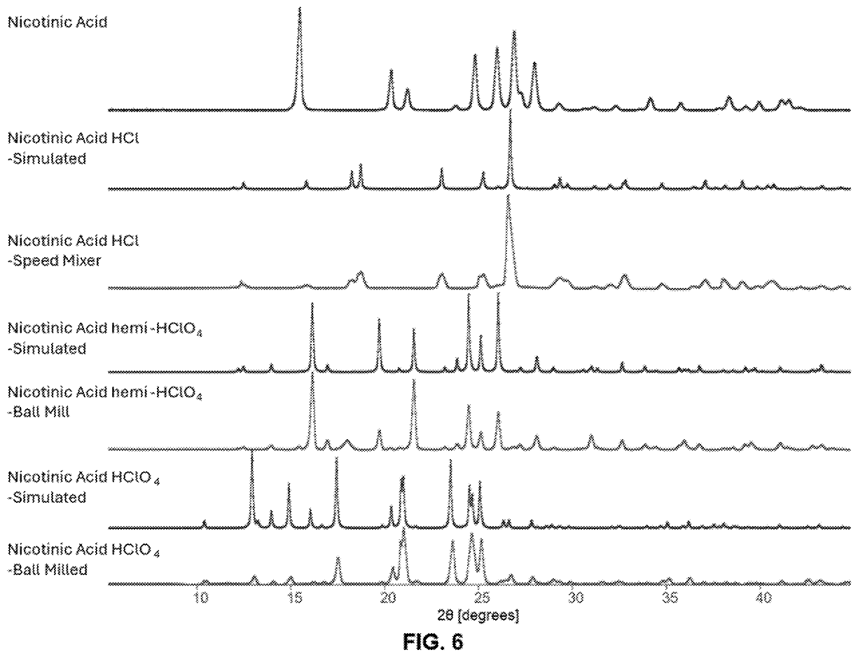

FIG. 7 shows the experimental and simulated PXRD patterns of isonicotinic acid and salts of isonicotinic acid.

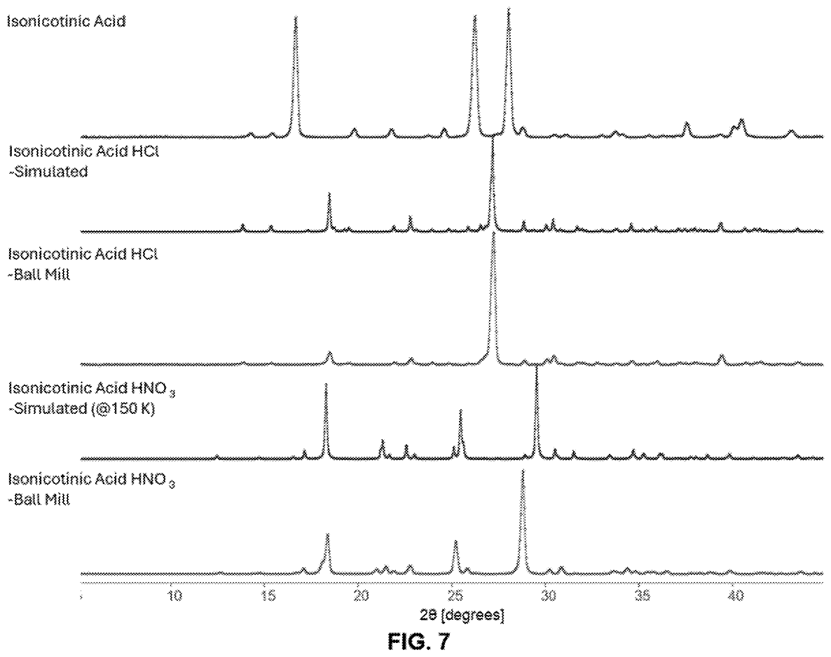

FIG. 8 shows the experimental and simulated PXRD patterns of picolinic acid and HCl salts of picolinic acid.

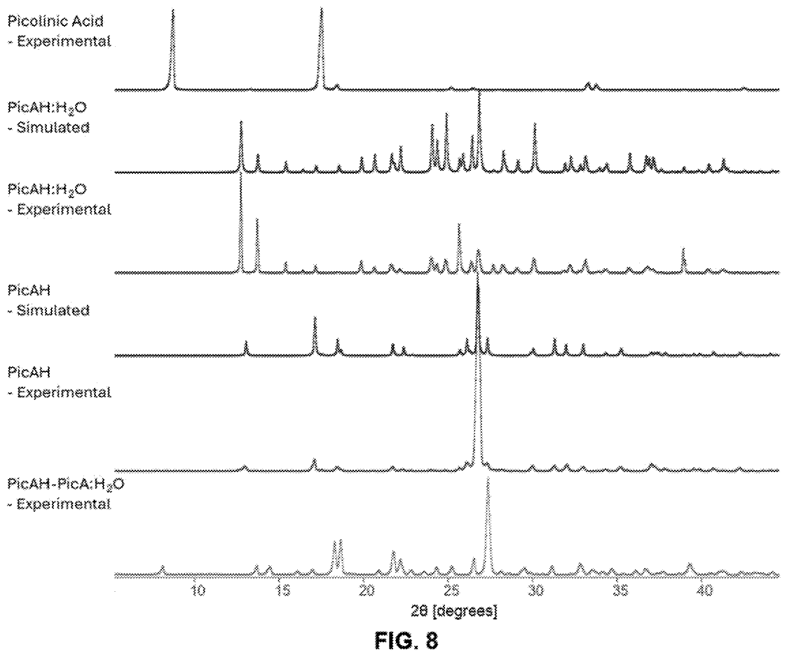

FIG. 9 shows the experimental and simulated PXRD patterns of caffeine and HCl salts of caffeine.

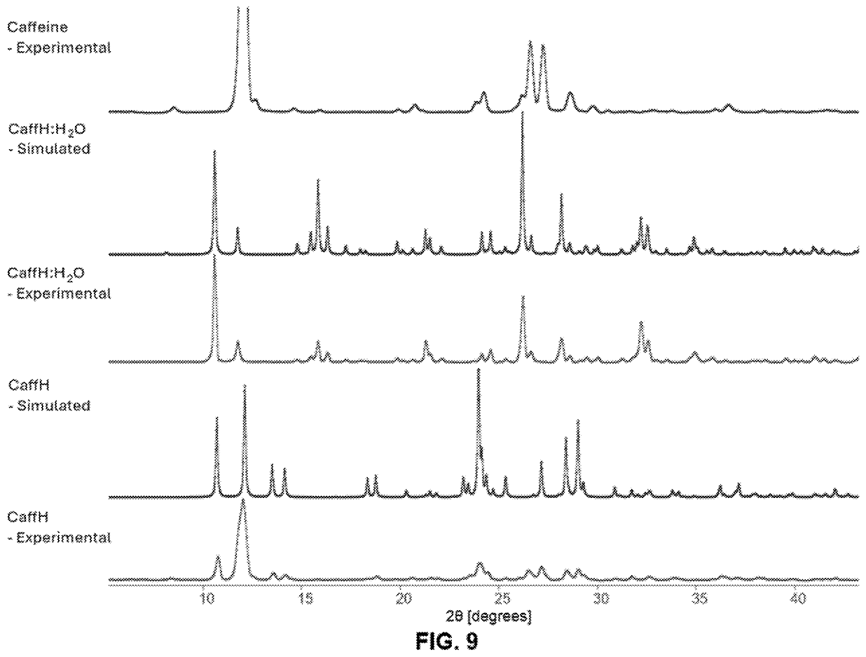

FIG. 10 shows the experimental and simulated PXRD patterns of theophylline, theobromine, and respective HCl salts.

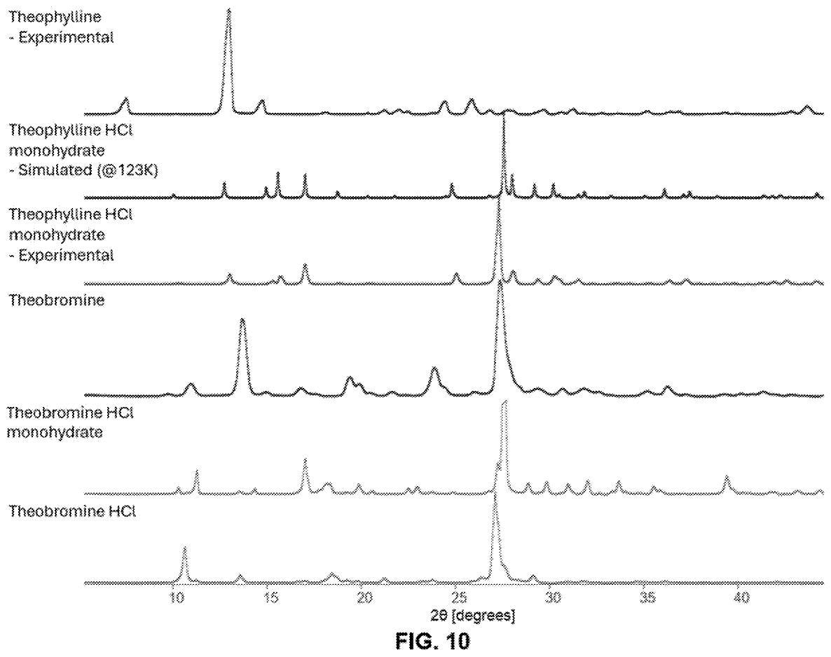

FIG. 11 shows the experimental and simulated PXRD patterns of acetaminophen and HCl salts of acetaminophen.

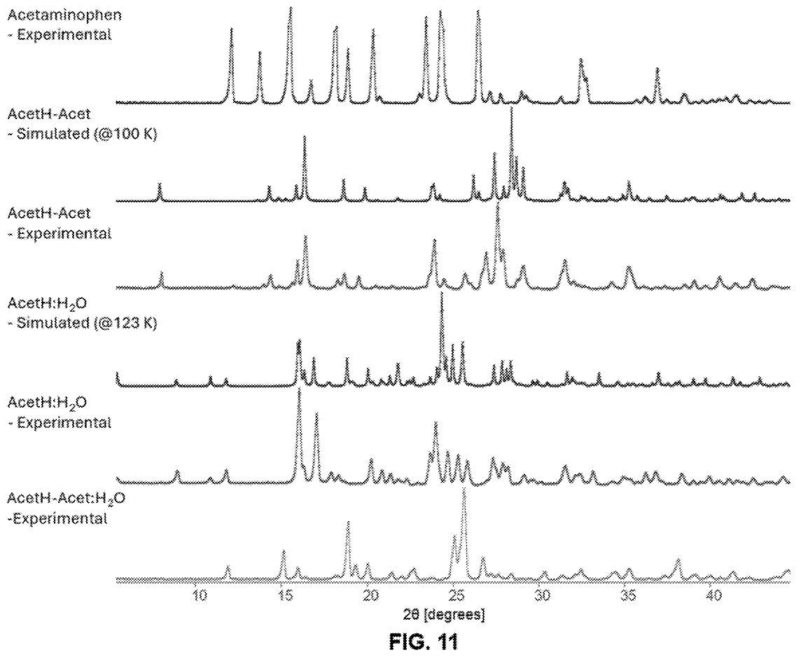

FIG. 12 shows the experimental and simulated PXRD patterns of metformin, creatine, and respective HCl salts.

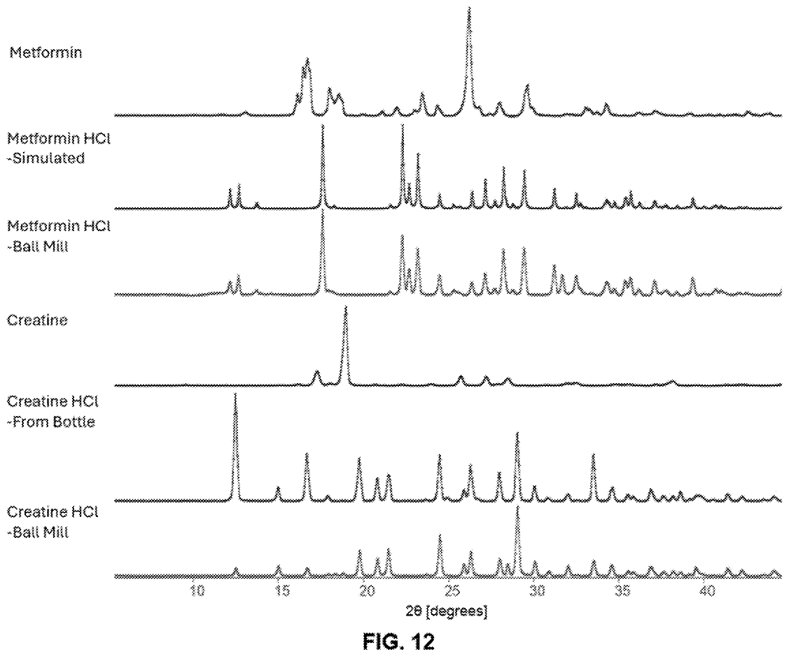

FIG. 13 shows the experimental and simulated PXRD patterns of L-Lysine and respective polyheterosalts of L-Lysine.

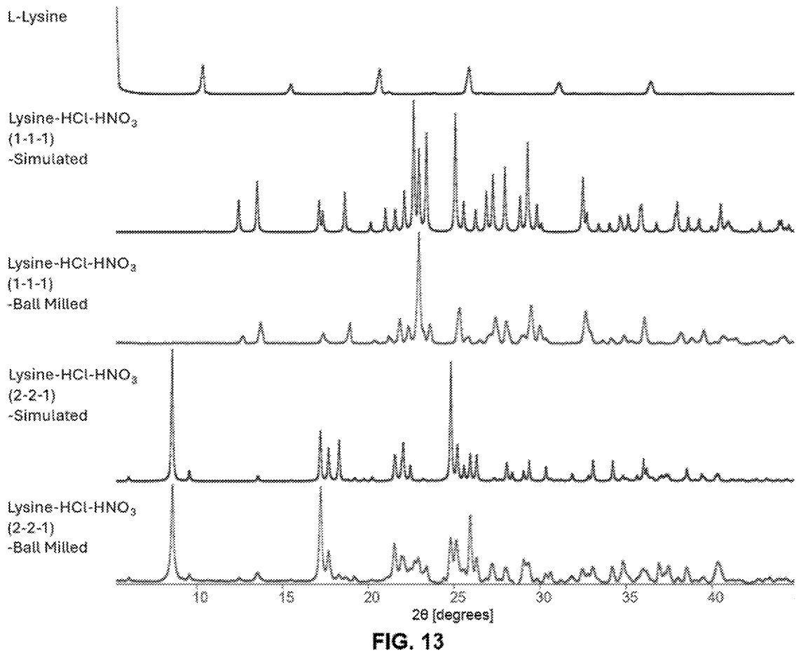

FIG. 14 shows the experimental and simulated PXRD patterns of L-Histidine and salts of L-Histidine.

FIG. 15 shows the experimental and simulated PXRD patterns of L-Alanine and L-Alanine HCl.

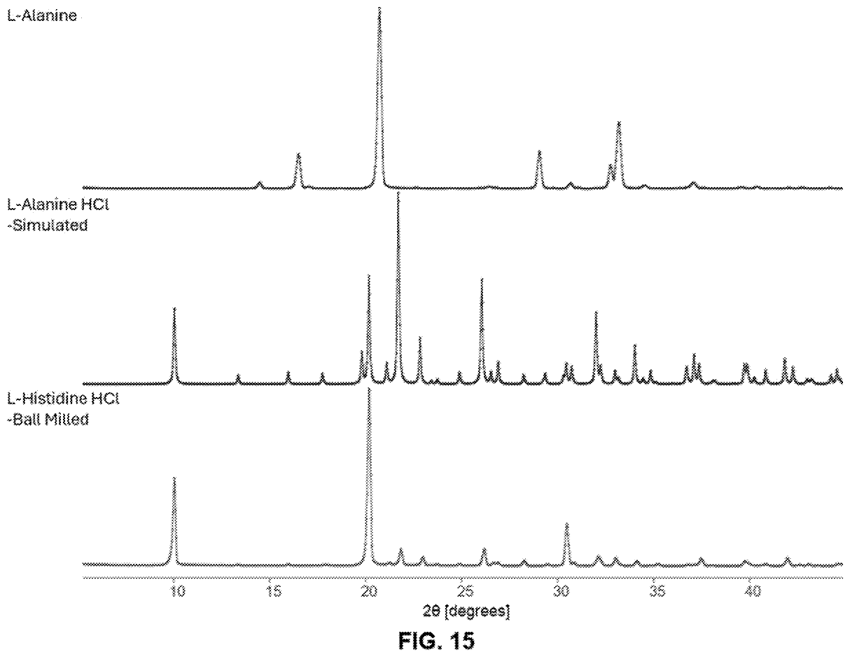

FIG. 16 shows the experimental and simulated PXRD patterns of sulfanilamide HCl.

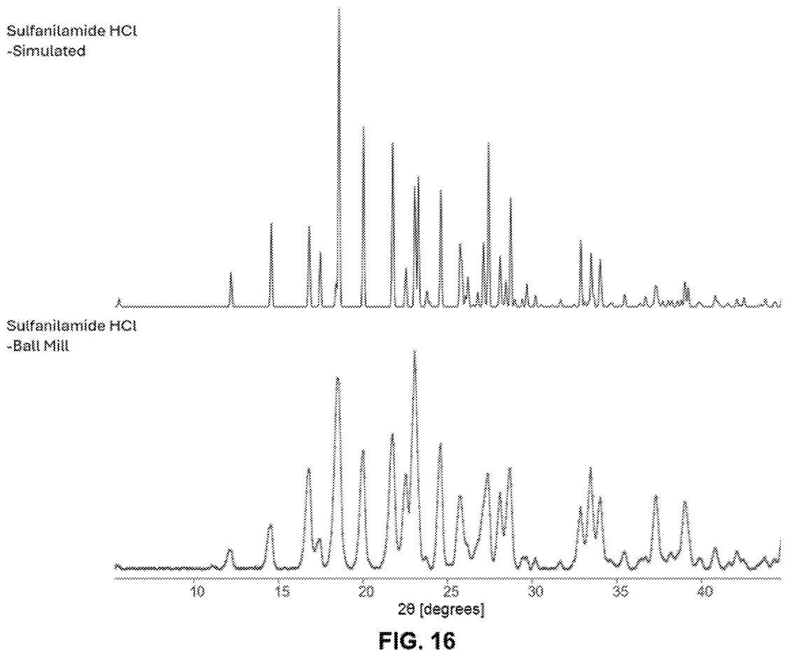

FIG. 17 shows the experimental and simulated PXRD patterns of isoniazid and salts of isoniazid.

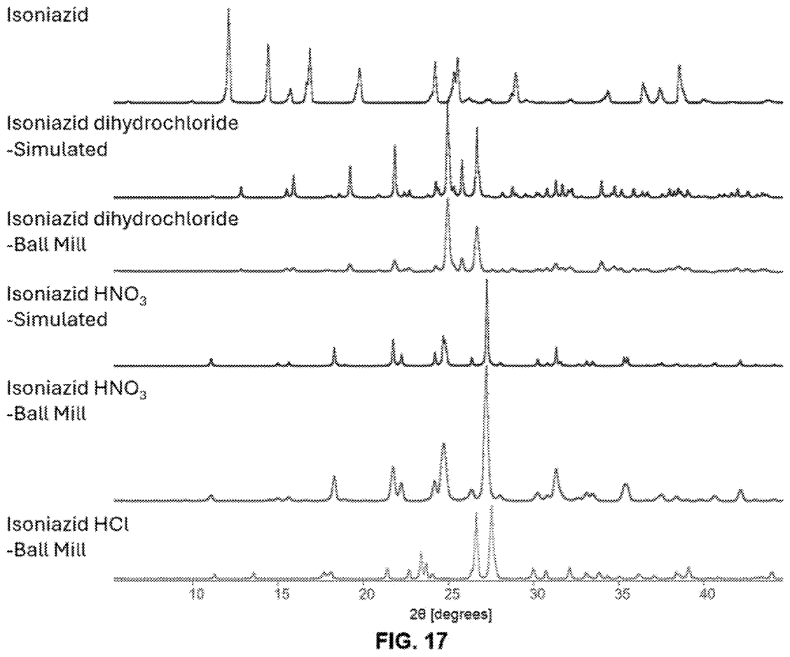

FIG. 18 shows the experimental and simulated PXRD patterns of amoxicillin trihydrate and amoxicillin trihydrate milled with 1 and 3 equivalents of HCl.

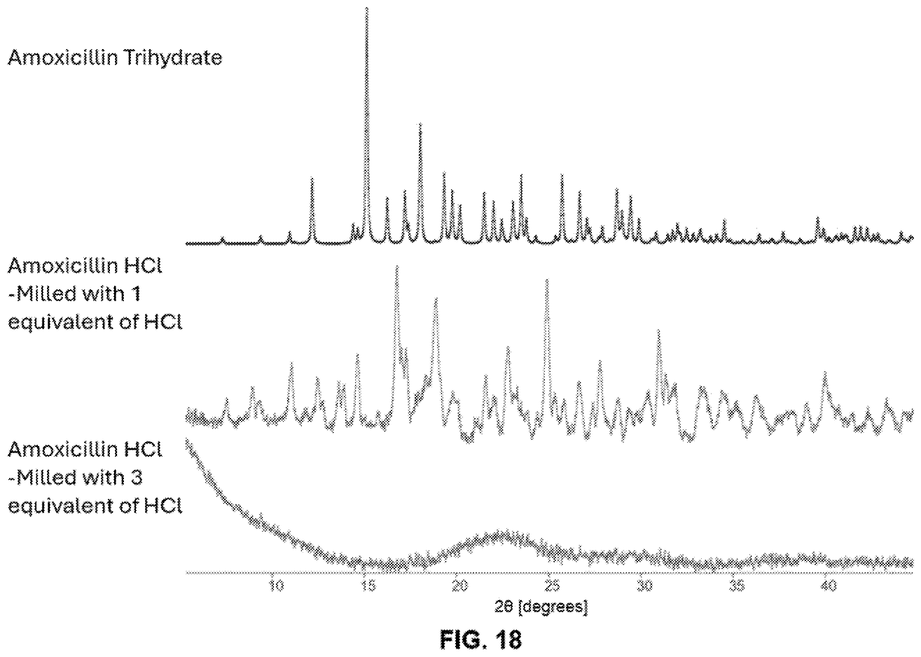

FIG. 19 shows ³⁵Cl static SSNMR spectra of NicH, NicH-Nic:H₂O, and NicAH acquired at 18.8 T.

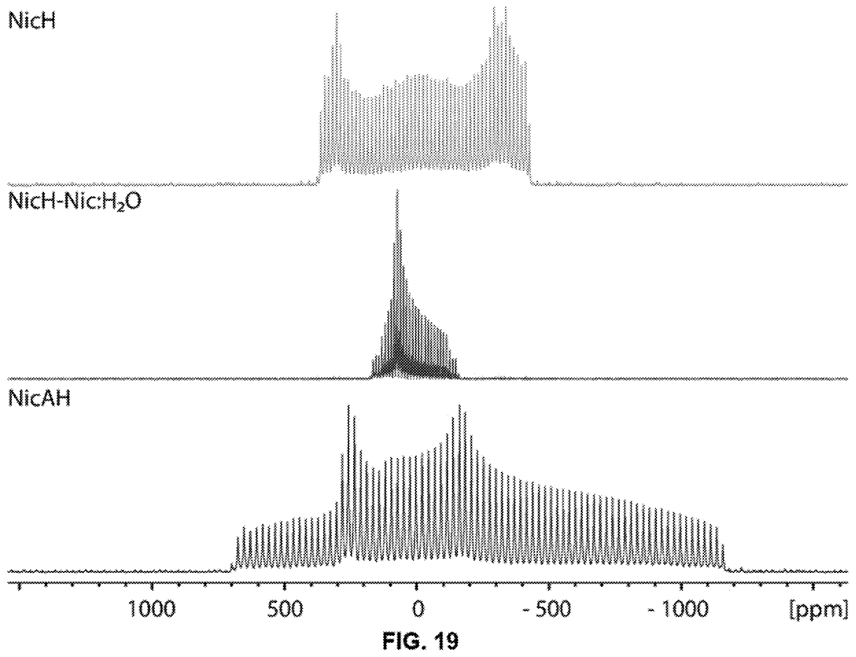

FIG. 20 shows ³⁵Cl static SSNMR spectra of IsoH, IsoH-Iso:H₂O, and IsoAH acquired at 18.8 T

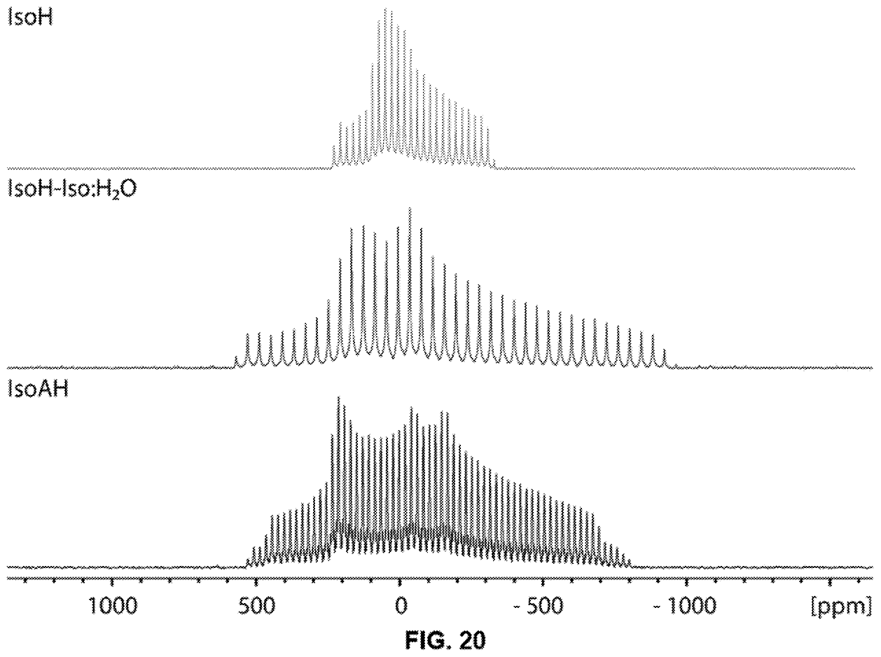

FIG. 21 shows ³⁵Cl static SSNMR of CaffH:H₂O and CaffH acquired at 18.8 T.

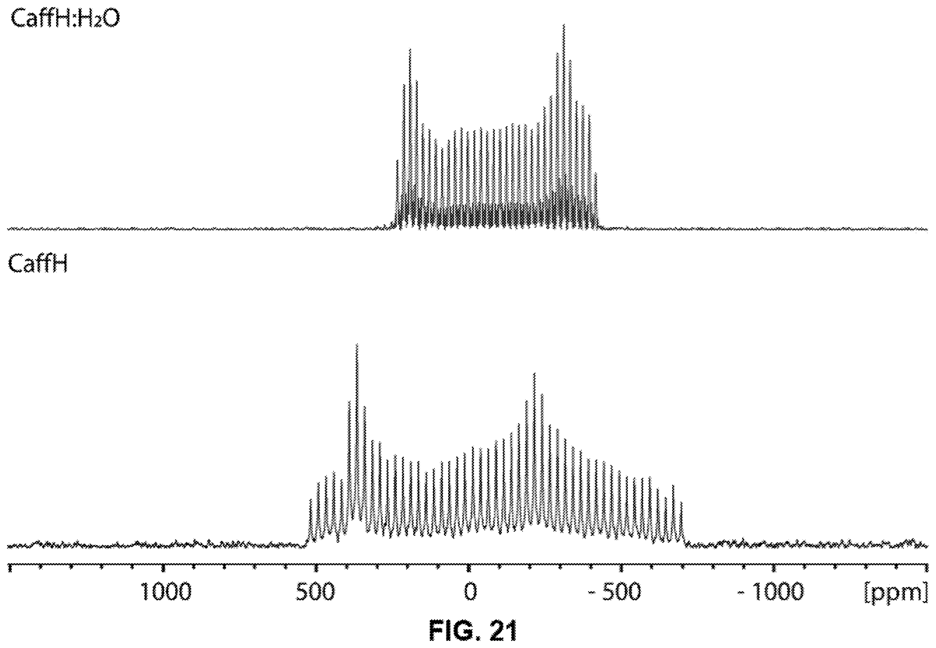

FIG. 22 shows the experimental and calculated PXRD patterns of Glu, GluHCl, GluHCl_{0.75}HBr_{0.25}, GluHCl_{0.50}HBr_{0.50}, GluHCl_{0.25}HBr_{0.75}, and GluHBr. The dashed yellow lines indicate key diffraction peaks that shift to the left as the concentration of Cl⁻ decreases. The dashed black line indicates a key diffraction peak in glutamic acid that is not present in any synthesized materials.

FIG. 23 shows the solid forms of various molecules formed with the methods described herein.

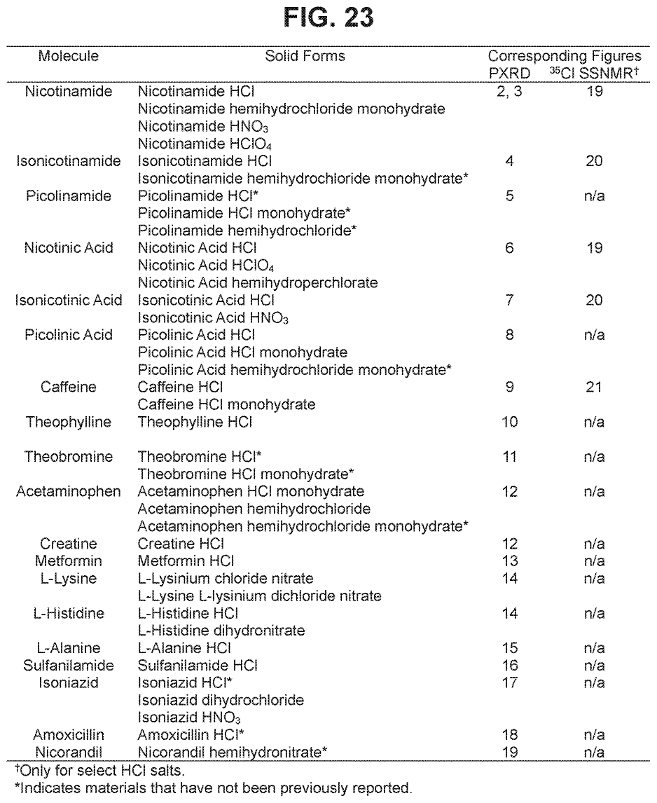

FIG. 24 shows the conditions and ball milling parameters that lead to quantitative syntheses of the salts of organic compounds.

FIG. 25 shows the results of competitive milling reactions involving two free bases and 12 M HCl(aq).

FIG. 26 shows a comparison of reported and idealized mechanochemical conditions with literature routes for hydrochloride salt formation

FIG. 27 shows ³⁵Cl EFG and chemical shift tensor parameters obtained from experimental measurements and DFT calculations on DFT-D2* geometry-optimized structures.

FIG. 28 shows the list of H . . . Cl⁻ contacts, associated moieties, and calculated ³⁵Cl SSNMR parameters for HCl salts, as determined from X-ray diffraction derived crystal structures.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

The term “a protonatable group” is a Lewis base that includes at least one pair of electrons capable of reacting with an acid such that the Leis base is protonated.

The term “a nitrogen bearing group” is a group that includes a nitrogen atom with a one pair of electrons capable of reacting with an acid such that the nitrogen atom is protonated.

The term “amino” is represented by the structure —NR¹R², where R¹ and R² are independently hydrogen, an alkyl group, an aryl group, or a cycloalkyl group.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic.

The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “nitrogen-bearing heteroaryl group” as used herein refers to an aromatic group that has at least one nitrogen atom incorporated within the ring of the aromatic group. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, imidazolyl, pyrimidinyl, tetrazolyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of nitrogen-bearing heteroaryl groups include, but are not limited to, pyridinyl, pyridazinyl, pyrimidinyl, pyrazinyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The term “secondary amide” is represented by the structure —C(O)NR¹R², where R¹ and R² are independently hydrogen, an alkyl group, an aryl group, or a cycloalkyl group.

The term “hydrazine group” is represented by the structure (R¹)₂N—N(R²)₂, where R¹ and R² are independently hydrogen, an alkyl group, an aryl group, or a cycloalkyl group. Alternatively, the hydrazine group can be included in a ring such as, for example, a cycloalkyl ring.

The term “guanidinyl group” is represented by the structure —N(R₁)—C(═N)—N(R²)₂, where R¹ and R² are independently hydrogen, an alkyl group, an aryl group, or a cycloalkyl group.

Unless otherwise specified, temperatures referred to herein are based on atmospheric pressure (i.e. one atmosphere).

### Methods for Producing Salts of Organic Molecules

Described herein are methods for producing a salt of a solid organic compound. The method involves (a) combining in the solid state the solid organic compound comprising one or more protonatable groups and an acid composition, wherein the amount of acid in the acid composition is about stoichiometric or less than stoichiometric relative to the one or more protonatable groups of the solid organic compound to produce a precursor mixture; and (b) mixing the precursor mixture by mechanochemical milling to produce the salt of the solid organic compound.

The first step involves selecting and combining in the solid state the solid organic compound and an acid composition. The solid organic molecule is any molecule that possess one or more protonatable groups, which is also referred to as a free base. Examples of such groups include, but are not limited to, an amino group, a nitrogen-bearing heteroaryl group, a secondary amido group, a hydrazine group, or a guanidinyl group. In one aspect, the organic molecule is a pharmaceutical, a nutraceutical, an agrochemical, or an explosive. In another aspect, the organic molecule is an amino acid, a peptide, a protein, a nucleic acid, or a small molecule. In another aspect, the organic molecule is nicotinamide, isonicotinamide, picolinamide, nicotinic acid, isonicotinic acid, picolinic acid, caffeine, theophylline, theobromine, acetaminophen, creatine, metformin, L-lysine, L-histidine, L-alanine, sulfanilamide, isoniazid, amoxicillin, nicorandil, amlodipine, sulfonamide, or pyrazinamide.

In one aspect, the acid used in the methods described herein can be an inorganic acid analog. Examples of inorganic acid analogs include, but are not limited to, a sulfonic acid (e.g., triflic acid, methanesulfonic acid, benezesulfonic acid, etc), a phosphonic acid, a boronic acid, or any combination thereof. In another aspect, the acid can be an inorganic acid such as, for example, H₂SO₄, HCl, HBr, HI, HClO₄, HNO₃, H₃PO₄, H₃PO₃, HF, HCN, H₂CrO₄, or any combination thereof.

The methods described herein do not use excessive amounts of acid to produce the salts, which prevent the formation of undesirable side products. In one aspect, the amount of acid in the acid composition is about stoichiometric or less than stoichiometric relative to each protonatable group of the solid organic compound. In one aspect, the molar ratio of the acid to stoichiometric or less than stoichiometric relative to each protonatable group of the solid organic compound is from 1:1 to 1:0.1, 1:1 to 1:0.2, 1:1 to 1:0.3, 1:1 to 1:0.4, 1:1 to 1:0.5, 1:1 to 1:0.6, 1:1 to 1:0.7, 1:1 to 1:0.8, or 1:1 to 1:0.9.

In certain aspects, when the solid organic compound includes two or more protonatable groups, it may be desirable to protonate only one protonatable group. In one aspect, when the solid organic compound possesses two or more protonatable groups, the amount of acid in the acid composition is about stoichiometric relative to a single protonatable group of the solid organic compound. For example, nicotinamide possesses two protonatable groups. When, 1:0.5 and 1:1 ratios of free base nicotinamide (Nic) to acid are used, NicH-Nic:H₂O and NicH are produced, respectively, as the protonation of the pyridyl nitrogen of nicotinamide is favorable.

The concentration of the acid in the acid composition can vary depending upon the protonatable group present in the solid organic compound. In one aspect, the concentration of the acid in the acid composition has a concentration of less than 15 M. In another aspect, the concentration of the acid in the acid composition has a concentration of 0.5 M, 1 M, 2 M, 3 M, 4 M, 5 M, 6 M, 7 M, 8 M, 9 M, 10 M, 11 M, 12 M, 13 M, 14 M, or less than 15 M, where any value can be a lower and upper endpoint of a range (e.g., 5 M to 13 M, etc.).

The methods described herein use acid compositions that have very little solvent (e.g., microliter scale relative to the amount of the organic molecule). Since so little solvent is used, the methods described herein are suitable for large scale manufacturing. In one aspect, the acid composition is less than about 10 μL per mg of the solid organic compound to produce a precursor mixture. In another aspect, the acid composition is from 0.1 μL per mg of the solid organic compound to less than about 10 μL per mg of the solid organic compound, or 0.1 μL, 0.5 μL, 1.0 μL, 1.5 μL, 2.0 μL, 2.5 μL, 3.0 μL, 3.5 μL, 4.0 μL, 4.5 μL, 5.0 μL, 5.5 μL, 6.0 μL, 6.5 μL, 7.0 μL, 7.5 μL, 8.0 μL, 8.5 μL, 9.0 μL, or 10.0 μL per mg of the solid organic compound, where any value can be a lower and upper endpoint of a range (e.g., 0.5 μL to 2.0 μL per mg of the solid organic compound). In another aspect, the weight ratio of the solid organic compound to the volume of the acid composition is from about 1:1 to about 4:1, or 1:1, 1.5:1, 2:1, 2.5:1, 3:1, 3.5:1, or 4:1, where any value can be a lower and upper endpoint of a range (e.g., 1.5:1 to 2.5:1, etc.).

In one aspect, the acid composition includes an acid and a solvent. In one aspect, the solvent is a polar solvent such as, for example, water, an alcohol (e.g., methanol, ethanol, etc.), or a combination thereof. In another aspect, the acid composition consists of an acid and a solvent such as, for example, comprises water, an alcohol (e.g., methanol, ethanol, etc.), or a combination thereof.

In one aspect, a cryogen is mixed with the solid organic compound and the acid composition prior to mechanochemical milling. The use of a cryogen can be employed to avert the buildup of heat during mechanochemical milling. For example, the solid organic compound and the acid composition when mixed are kept at temperature below room temperature to control the final product to be produced (e.g., formation of a hydrate vs. no hydrate).

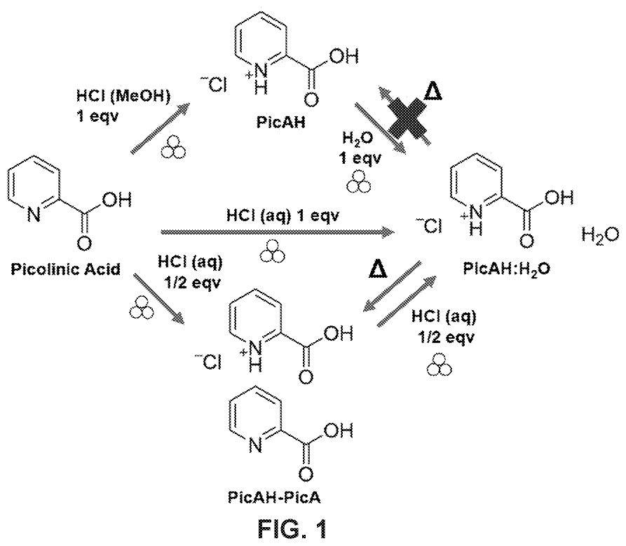

Depending upon the solvent used to produce the acid composition, the salt of the solid organic compound can be an anhydrous compound or a monohydrate compound. For example, if a 1:1 molar ratio of HCl in water and picolinic acid (PicA), caffeine (Caff), and acetaminophen (Acet) are selected, the hydrates picolinic acid HCl monohydrate (PicAH:H₂O), caffeine HCl monohydrate (CaffH:H₂O), and acetaminophen HCl monohydrate (AcetH:H₂O) are formed (FIG. 1). Alternatively, if similar reactions are conducted with HCl (MeOH) or HCl (EtOH), the anhydrous salts picolinic acid HCl (PicAH), caffeine HCl (CaffH), and acetaminophen hemihydrochloride (AcetH-Acet) are formed (FIG. 1). While synthesizing a simple salt is relatively straightforward, forming a hemihydrochloride salt can be more challenging. The methods described herein permit the synthesis of hemihydrate salts.

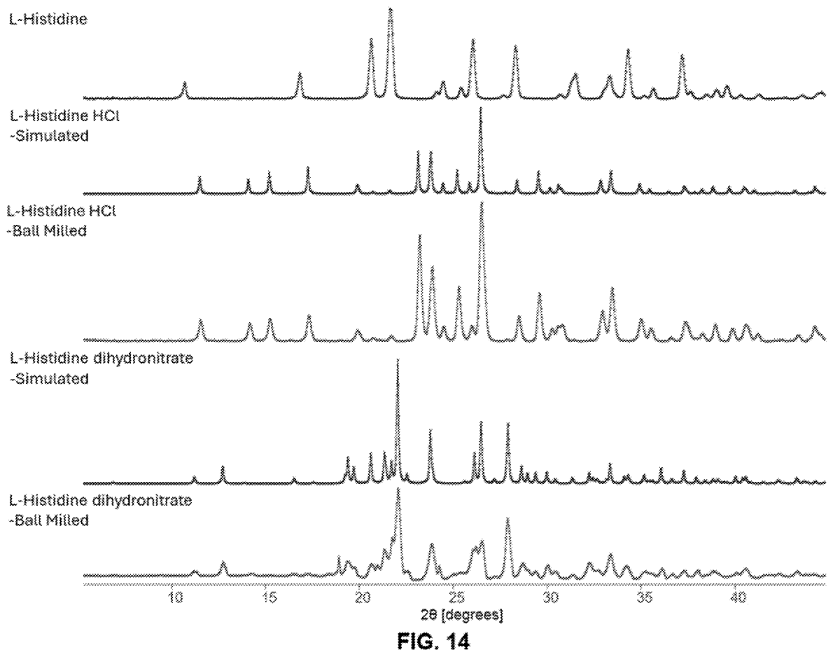

In one aspect, the acid composition can include two or more two or more different acids to produce polyheterosalts or mixed salts, which are generally defined as chemically homogeneous salts characterized by the presence of multiple distinct ions within a single, well-defined stoichiometric compound. For example, a specific mixed salt like L-lysinium L-lysine dichloride nitrate (LysH-Lys-Cl₂/NO₃) or L-lysinium chloride nitrate (LysH—Cl/NO₃) in high purity (e.g., FIG. 14) by altering the amount of each acid used using the methods described herein. Mixed salts can be relevant in pharmaceuticals where the presence of two distinct anions (e.g., Cl⁻ and NO₃⁻, Cl⁻ and Br⁻, etc.) may favorably modify the physicochemical properties (e.g., solubility, bioavailability, stability, etc.) of the solid form of the API. Furthermore, mixed salts of organic compounds can also exhibit unique electrochemical or optical properties, which could be of great value in the design of novel materials with advanced functionalities.

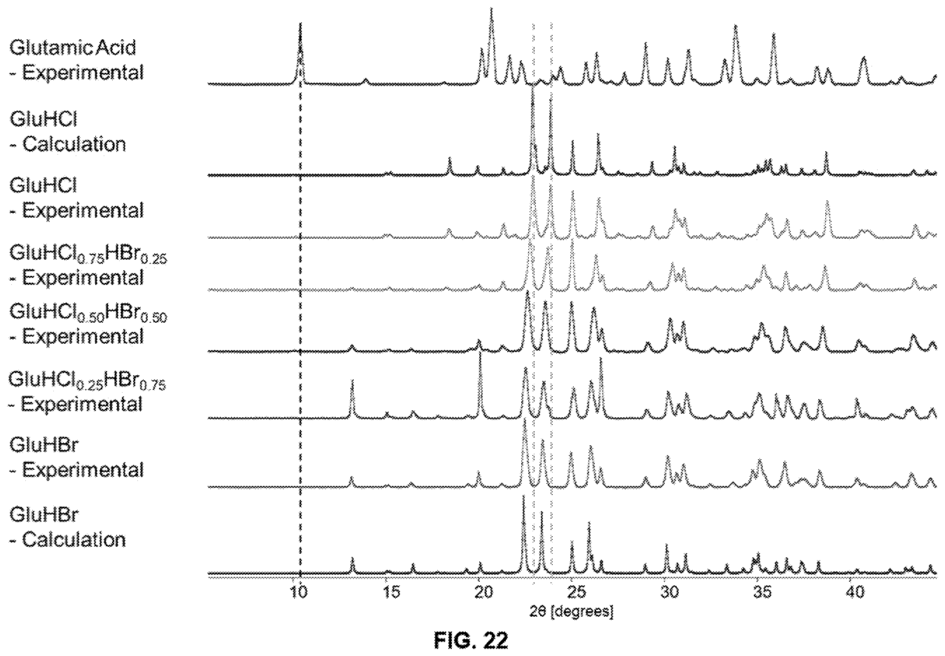

In one aspect, the molar ratio of the sum of the two or more acids is about stoichiometric relative to each protonatable group of the solid organic compound. For example, if the acid composition includes HCl and HBr, the amount of HCl and HBr can be varies such that the molar sum of acid is stoichiometric equivalent to each protonatable group (e.g., 50% HCl/50% H Br, 25% HCl/75% HBr, 75%% HCl/25% HBr, etc.). The Examples provide non-limiting procedures for making polyheterosalts, with an example depicted in FIG. 22.

The solid organic compound and the acid composition are mixed by mechanochemical milling. Mechanochemical milling involves techniques that use mechanical force to drive chemical reactions, typically without the need for solvents or heat. These methods rely on mechanical energy to induce chemical reactions, often leading to faster reaction rates, unique products, and more environmentally friendly processes compared to traditional solution-based chemistry. Examples of such techniques include, but are not limited to, ball milling, resonant acoustic mixing, speed mixing, extrusion, planetary milling, or grinding via mortar and pestle or automated mills.

When conducting mechanochemical milling, the duration and frequency of the milling (i.e., mixing) can be varied. Generally, a sufficient duration is necessary to allow the reaction products to reach an equilibrium state, and higher milling/mixing frequencies speed up the reaction. The milling/mixing frequency can be modified to avoid temperature increases that could adversely affect the reaction between the acid and solid organic molecule. In addition to milling/mixing frequency, the material and volume that is mixed/milled and the nature and size of ball bearings can be modified to control the temperature during mechanochemical milling.

In one aspect, a precursor mixture composed of the solid organic compound and the acid composition is mixed by mechanochemical milling for about 1 minute to about 30 minutes, or 1 minute, 3 minutes, 6 minutes, 9 minutes, 12 minutes, 15 minutes, 18 minutes, 21 minutes, 24 minutes, 27 minutes, or 30 minutes, where any value can be a lower and upper endpoint of a range (e.g., 9 minutes to 15 minutes).

In one aspect, a precursor mixture composed of the solid organic compound and the acid composition is mixed by mechanochemical milling at a temperature of about 20° C. to about 25° C., 20° C., 21° C., 22° C., 23° C., 24° C., or 25° C., where any value can be a lower and upper endpoint of a range (e.g., 21° C. to 23° C.).

In one aspect, a precursor mixture composed of the solid organic compound and the acid composition is mixed by mechanochemical milling at a frequency of about 10 Hz to about 100 Hz, or 20 Hz, 25 Hz, 30 Hz, 35 Hz, 40 Hz, 45 Hz, 50 Hz, 60 Hz, 70 Hz, 80 Hz, 90 Hz, or 100 Hz, where any value can be a lower and upper endpoint of a range (e.g., 25 Hz to 35 Hz).

In another aspect, mechanochemical milling is performed by liquid assisted grinding (LAG). LAG is mechanochemical method that involves adding a small amount of liquid during grinding or ball milling. The liquid acts as a catalytic medium, enhancing the efficiency of energy transfer between particles without fully dissolving the reactants. The liquid may also be incorporated into the product by forming a solvate. In one aspect, when the acid composition includes a solvent such as water or an alcohol, the solvent can be used to assist the mechanochemical milling of the solid organic compound and the acid of the acid composition.

In another aspect, a salt of a solid organic compound can be produced by the method comprising:
mixing a solid organic compound comprising one or more protonatable groups and an acid composition, wherein the amount of acid in the acid composition is about stoichiometric or less than stoichiometric relative to the one or more protonatable groups of the solid organic compound to produce a precursor mixture to produce a liquid composition where the solid organic compound is dissolved in the acid composition; and
evaporating the liquid from the liquid composition to produce the salt of the solid organic compound.

In this aspect, any of the solid organic compounds and acid compositions described above can be used in this method. In one aspect, the solid organic compound and acid composition are mixed so that the solid organic compound is dissolved in the acid composition. In one aspect, the solid organic compound and acid composition are heated to promote the dissolution of the solid organic compound into the acid composition. After the liquid composition of the composed of the solid organic compound into the acid composition, the liquid in the liquid composition is removed by evaporation. Techniques know in the art for removing the liquid from the liquid composition can be used herein. In one aspect, the liquid is removed slow evaporation, where the liquid composition is allowed to stand at ambient temperature for a sufficient time such that the liquid evaporates to produce the salt of the solid organic compound.

The methods described herein possess several advantages when compared to compared to conventional techniques for making salts. The methods described herein use at most stoichiometric amounts of acid, often using the most concentrated reagent possible, which greatly reduces both solvent waste and cost. Moreover, using only stoichiometric amounts of the acid is far safer than solvent-based reactions, since dissolution is not required (microliters vs. milliliters on lab scale translates to milliliters vs. liters on a larger scale). For example, to produce organic HCl salts, the methods described herein do not require the material to be dissolved in any solvent.

In many cases, slow evaporation and recrystallization can take anywhere from days to months to produce the salts. The methods described herein permit the production of organic salts (microcrystalline to amorphous) in between 1-30 minutes. Undesired side reactions are less likely to occur using the methods described herein, as there is no excess of acid facilitating acid hydrolysis reactions and/or polymorphic crystallization due to spatial heterogeneity (i.e., temperature or concentration gradients).

Finally, since so little solvent is used, the methods described herein are eminently suitable for large scale manufacturing. At an industrial scale, the methods described herein represent a great potential for monetary savings and increased efficiency, benefiting both the manufacturer and consumer, since it requires very little solvent and energy input (i.e., no heating), reduces solvent waste and impurities, and nets almost quantitative yields (i.e., atom economy). The methods can also be employed for the “on-demand” production of pharmaceuticals that maybe unstable, allowing for in-house investigations of new solid forms and the capability to rapidly produce dosage forms where the API is stabilized. This is particularly advantageous when another property, such as exceptional bioavailability, makes the salt highly desirable despite its limited stability.

### ASPECTS

Aspect 1. A method for producing a salt of a solid organic compound, the method comprising:
combining in the solid state the solid organic compound comprising one or more protonatable groups and an acid composition, wherein the amount of acid in the acid composition is about stoichiometric or less than stoichiometric relative to the one or more protonatable groups of the solid organic compound to produce a precursor mixture; and
mixing the precursor mixture by mechanochemical milling to produce the salt of the solid organic compound.

Aspect 2. The method of Aspect 1, wherein the acid comprises an inorganic acid analog.

Aspect 3. The method of Aspect 2, wherein the inorganic acid analog comprises a sulfonic acid, a phosphonic acid, a boronic acid, or any combination thereof.

Aspect 4. The method of Aspect 1, wherein the acid comprises an inorganic acid.

Aspect 5. The method of Aspect 4, wherein the inorganic acid comprises H₂SO₄, HCl, HBr, HI, HClO₄, HNO₃, H₃PO₄, H₃PO₃, HF, HCN, H₂CrO₄, or any combination thereof.

Aspect 6. The method of any one of Aspects 1-5, wherein the acid composition is less than about 10 μL per mg of the solid organic compound.

Aspect 7. The method of any one of Aspects 1-5, wherein the acid composition is from 0.1 μL per mg of the solid organic compound to about 10 μL per mg.

Aspect 8. The method of any one of Aspects 1-7, wherein the acid composition comprises a polar solvent.

Aspect 9. The method of Aspect 8, wherein the polar solvent comprises water, an alcohol, or a combination thereof.

Aspect 10. The method of Aspect 1, wherein the acid composition consists of the acid and a polar solvent.

Aspect 11. The method of Aspect 1, wherein the acid composition comprises HCl and a polar solvent comprising methanol, ethanol, water, or any combination thereof.

Aspect 12. The method of any one of Aspects 1-11, wherein the acid composition has a concentration of less than 15 M.

Aspect 13. The method of any one of Aspects 1-11, wherein the acid composition has a concentration of 0.5 M to less than 15 M.

Aspect 14. The method of any one of Aspects 1-13, wherein a ratio of a weight of the solid organic compound to a volume of the acid composition is from about 1:1 to about 4:1.

Aspect 15. The method of any one of Aspects 1-14, wherein the mechanochemical milling comprises ball milling, resonant acoustic mixing, speed mixing, extrusion, planetary milling, or grinding via mortar and pestle or an automated mill.

Aspect 16. The method of any one of Aspects 1-15, wherein the precursor mixture is mixed by mechanochemical milling for about 1 minute to about 30 minutes.

Aspect 17. The method of any one of Aspects 1-16, wherein the precursor mixture is mixed by mechanochemical milling at a temperature less than or equal to 25° C.

Aspect 18. The method of any one of Aspects 1-16, wherein the precursor mixture is mixed by mechanochemical milling at a temperature of about 20° C. to about 25° C.

Aspect 19. The method of any one of Aspects 1-18, wherein the precursor mixture is mixed by mechanochemical milling at a frequency of about 10 Hz to about 100 Hz.

Aspect 20. The method of any one of Aspects 1-19, wherein the protonatable group comprises a nitrogen bearing group.

Aspect 21. The method of any one of Aspects 1-20, wherein the protonatable group comprises an amino group, a nitrogen-bearing heteroaryl group, a secondary amido group, a hydrazine group, or a guanidinyl group.

Aspect 22. The method of any one of Aspects 1-21, wherein the solid organic compound comprises a pharmaceutical, a nutraceutical, an agrochemical, or an explosive.

Aspect 23. The method of any one of Aspects 1-21, wherein the solid organic compound comprises an amino acid, a peptide, a protein, a nucleic acid, or a small molecule.

Aspect 24. The method of any one of Aspects 1-21, wherein the solid organic compound comprises nicotinamide, isonicotinamide, picolinamide, nicotinic acid, isonicotinic acid, picolinic acid, caffeine, theophylline, theobromine, acetaminophen, creatine, metformin, L-lysine, L-histidine, L-alanine, sulfanilamide, isoniazid, amoxicillin, nicorandil, amlodipine, sulfonamide, or pyrazinamide.

Aspect 25. The method of any one of Aspects 1-24, wherein the acid composition comprises two or more different acids.

Aspect 26. The method of any one of Aspects 1-24, wherein the amount of acid in the acid composition is about stoichiometric relative to each protonatable group of the solid organic compound.

Aspect 27. The method of any one of Aspects 1-24, wherein when the solid organic compound possesses two or more protonatable groups, the amount of acid in the acid composition is about stoichiometric relative to a single protonatable group of the solid organic compound.

Aspect 28. The method of any one of Aspects 1-27, wherein the salt of the solid organic compound is an anhydrous compound.

Aspect 29. The method of any one of Aspects 1-27, wherein the salt of the solid organic compound is a monohydrate compound.

Aspect 30. A salt of a solid organic compound produced by the method of any one of Aspects 1-29.

Aspect 31. A salt of a solid organic compound comprising isonicotinamide-isonicotinamidium chloride monohydrate (Iso-IsoH:H₂O), picolinamide HCl monohydrate (PicH:H₂O), picolinamide HCl (PicH), picolinamide-picolinamidium chloride (PicH-Pic), picolinic acid hemihydrochloride (PicAH-PicA), and (bis)acetaminophen HCl monohydrate (Acet-AcetH:H₂O).

Aspect 32. A method for producing a salt of a solid organic compound, the method comprising:
mixing a solid organic compound comprising one or more protonatable groups and an acid composition, wherein the amount of acid in the acid composition is about stoichiometric or less than stoichiometric relative to the one or more protonatable groups of the solid organic compound to produce a precursor mixture to produce a liquid composition where the solid organic compound is dissolved in the acid composition; and
evaporating the liquid from the liquid composition to produce the salt of the solid organic compound.

Aspect 33. A salt of a solid organic compound produced by the method of Aspect 32.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### EXPERIMENTAL

### Materials

Nicotinic acid (NicA), isonicotinic acid (IsoA), picolinic acid (PicA), acetaminophen (Acet), nicotinamide (Nic), isonicotinamide (Iso), picolinamide (Pic), and caffeine (Caff) (Scheme 1) were purchased from MilliporeSigma and used without further purification. These reagents were used to mechanochemically synthesize eleven previously-reported salts, including: nicotinamide hemihydrochloride hydrate (NicH-Nic:H₂O), nicotinamide HCl (NicH), isonicotinamide HCl (IsoH), nicotinic acid HCl (NicAH), isonicotinic acid HCl (IsoAH), picolinic acid HCl monohydrate (PicAH:H₂O), anhydrous picolinic acid HCl (PicAH), caffeine HCl dihydrate (CaffH:H₂O), caffeine HCl (CaffH), bis(acetaminophen) HCl (AcetH-Acet), and acetaminophen HCl monohydrate (AcetH:H₂O).⁷⁻¹⁵ In addition, they were also used to produce six novel salts, including: isonicotinamide-isonicotinamidium chloride monohydrate (Iso-IsoH:H₂O), picolinamide HCl monohydrate (PicH:H₂O), picolinamide HCl (PicH), picolinamide-picolinamidium chloride (PicH-Pic), picolinic acid hemihydrochloride (PicAH-PicA), and (bis)acetaminophen HCl monohydrate (Acet-AcetH:H₂O).

### Synthesis of HCl Salts

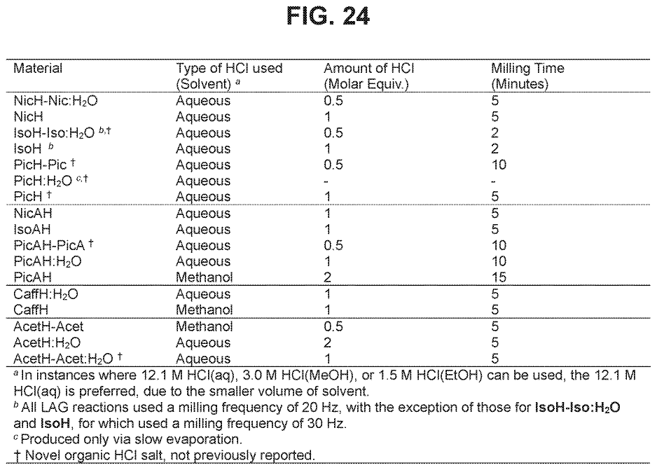

HCl salts were synthesized using slow evaporation and/or mechanochemical grinding. Mechanochemical syntheses were carried out under a variety of conditions using 12.1 M HCl(aq), 3.0 M methanolic HCl (HCl(MeOH)), or 1.25 M ethanolic HCl (HCl(EtOH)) as milling liquids. Ball milling reactions used 10 mL Teflon milling jars with one 10 mm Teflon ball bearing and were conducted using a Retsch Mixer Mill 500 Vario with a milling frequency ranging from 20 to 35 Hz, and milling times ranging from 2 to 15 min (unless specified otherwise). For larger scale reactions conducted with 75 or 100 g of solids, a FlackTek Model 330-100 SE speed mixer was used with 250 mL polypropylene sample holders at 2000 RPM for 2 minutes. The optimized milling conditions for efficient, high-yield, lab-scale syntheses are presented in FIG. 24. No further grinding of reaction products was needed prior to analyses by SSNMR or PXRD. For syntheses via slow evaporation, a solution containing one organic reagent was dissolved in a 20 mL glass vial with gentle heating at ca. 55° C. to completely dissolve the solids. The solutions were allowed to evaporate for several days to weeks under ambient conditions. Suitable crystals were used for analysis by SCXRD.

### Powder X-ray Diffraction

PXRD patterns were acquired on a Rigaku Miniflex with a Cu Kα (λ=1.540593 A) radiation source and a HyPix-400 MF 2D hybrid pixel array detector. The X-ray tube voltage and amperage were set to 40 kV and 15 mA, respectively. Experiments were run with the detector scanning 2θangles from 2.5° to 50° with a step size of 0.03° at a speed of 5° min⁻¹. The PXRD patterns for all reagents and cocrystals were compared to patterns simulated from reported structural models^{7-15,71-80} using the CrystalDiffract⁸¹ software package to confirm crystallinity and purity, and to identify novel phases.⁸¹

### Single-Crystal X-Ray Diffraction

A clear light yellow, irregular shaped crystal of PicH:H₂O was mounted on a nylon loop with perfluoroether oil. The sample was crystallized by a diffusion method. Data were collected from a shock-cooled single crystal at room temperature on a XtaLAB Synergy, Dualflex, HyPix four-circle diffractometer with a micro-focus sealed X-ray tube using a mirror as monochromator and a HyPix detector. The diffractometer was equipped with an Oxford Cryostream 800 low-temperature device and used Cu Kα radiation (λ=1.54184 Å). All data were integrated with Crysalispro and a gaussian absorption correction using SCALE3 ABSPACK was applied. The structure was solved by dual methods using SHELXT⁸² and refined by full-matrix least-squares methods against F² by SHELXL.⁸³ All non-hydrogen atoms were refined with anisotropic displacement parameters. All C-bound hydrogen atoms were refined isotropic on calculated positions using a riding model with their Uᵢₛₒ values constrained to 1.5 times the U_{eq} of their pivot atoms for terminal sp³ carbon atoms and 1.2 times for all other carbon atoms. Disordered moieties were refined using bond lengths restraints and displacement parameter restraints. Crystallographic data for the structure reported in this paper has been deposited with the Cambridge Crystallographic Data Centre under the deposition number 2341820.

### Thermogravimetric Analysis

Thermogravimetric analysis (TGA) measurements were performed on a TA Instruments TGA 550 with samples packed in alumina crucibles. Samples were heated from room temperature (~23° C.) to 400° C. at a heating rate of 10° C. min⁻¹ under a dry argon purge (gas flow of 25 ml min⁻¹).

### Solid-State NMR Spectroscopy

Overview. SSNMR experiments were conducted at the National High Magnetic Field Laboratory (NHMFL) in Tallahassee, FL. Experiments at B₀=18.8 T [v₀(¹H)=800.13 MHz, v₀(³⁵Cl)=78.42 MHz, and v₀(³⁷Cl)=65.35 MHz] were conducted on Bruker Avance NEO spectrometer with an Oxford standard bore magnet. Magic-angle spinning (MAS) and static (stationary sample) experiments were carried out with NHMFL-built 5.0 mm HX or 3.2 mm HXY probes, with samples packed into 3.2 zirconia oxide rotors (³⁵Cl static/MAS) or 5.0 mm o.d. polychlorotrifluoroethylene sample holders (³⁷Cl static).

^{35/37}Cl SSNMR. Static ³⁵Cl{¹H} SSNMR spectra were obtained at 18.8 T using a variety of pulse sequences (Hahn Echo,⁸⁴ QCPMG,⁸⁵⁻⁸⁷ CP-CPMG,⁸⁸⁻⁹⁰ and WURST-CPMG⁹¹⁻⁹³) with a continuous wave (CW) ¹H decoupling field of 50 kHz. All WURST pulses used a 500 kHz sweep width. ³⁷Cl{¹H} static spectra were obtained using Hahn Echo, QCPMG, or CP-CPMG pulse sequences, with CW ¹H decoupling field of 50 kHz. ³⁶Cl{¹H} MAS spectra were obtained using a Bloch decay or rotor-synchronized Hahn Echo or QCPMG pulse sequence, with an MAS rate of vᵣₒₜ=10-20 kHz and 50 kHz of SPINAL-64 deocupling.^{94-96 35/37}Cl chemical shifts were referenced to 1.0 M NaCl(aq) (δᵢₛₒ=0.0 ppm) using NaCl(s) (δᵢₛₒ=−41.1 ppm) as a secondary reference.

Spectral Processing and Simulation. Spectra were processed using the TopSpin v.3.7 software package. Simulations of ^{35/37}Cl SSNMR spectra were generated using the ssNake v.1.5 software package.⁹⁷ Euler angles extracted from simulations in ssNake, which use the ZX′Z″ convention, were converted to the ZY′Z″ convention and verified through additional simulations in WSolids^{98,99}

### DFT Calculations

Calculations were performed on structural models based on previous SCXRD studies,^{7-15,71,100} with the exception of PicH:H₂O, for which the structural model is based on the SCXRD structure reported herein. Geometry optimizations and subsequent calculations of ³⁵Cl EFG and magnetic shielding tensors were performed using dispersion-corrected plane-wave DFT methods (DFT-D2*)^{101,102} using the CASTEP module^{103,104} within Materials Studio 2020. These calculations used the generalized gradient approximation (GGA) with the RPBE functional,¹⁰⁵ ultrasoft pseudopotentials generated on-the-fly,¹⁰⁶ a k-point spacing of 0.05 Å⁻¹, a plane-wave cut-off energy of 800 eV, and an SCF convergence threshold of 5×10⁻⁷ eV. Geometry optimizations employed the LBFGS method,¹⁰⁷ in which the positions of all atoms were relaxed with unit cell parameters fixed at their experimental values. Thresholds for structural convergence included a maximum change in energy of 5×10⁻⁶ eV atom⁻¹, a maximum displacement of 5×10⁻⁴ Åatom⁻¹, and a maximum Cartesian force of 10⁻² eV Å⁻¹. A correction to the dispersion energy was introduced using Grimme's semi-empirical two-body dispersion force field,^{108,109} with an empirical reparameterization introduced by our group.^{101,102,110 35}Cl magnetic shielding tensors were calculated using the GIPAW method.^{111 35}Cl chemical shift values were referenced with respect to that of NaCl(s) (δᵢₛₒ=−41.1 ppm), which has an absolute isotropic magnetic shielding value of 993.28 ppm. EFGShield¹¹² was used to extract the Euler angles describing the relative orientations of magnetic shielding and EFG tensors, which are reported using the ZYZ″ convention for rotation.⁹⁹

RESULTS AND DISCUSSION

### Overview

The outcomes of all mechanochemical syntheses were assessed using PXRD data, with patterns acquired for all previously reported and novel organic HCl salts. Those of the former are in good agreement with simulated patterns based on their crystal structures, while those of the latter are compared to patterns of educts, with distinct diffraction peaks attributed to novel phases (based on this, the stoichiometry of the reaction, and no detection of impurities via PXRD and/or other analytical techniques, vide infra). The PXRD pattern of PicH:H₂O was also compared to simulated patterns derived from the novel crystal structure reported herein and found to be in agreement. These results confirm that the syntheses are successful, and the products are crystalline with no presence of impurities.

### Mechanochemical Salification Methods for the Synthesis of Organic HCl Salts

Eleven known and five novel organic HCl salts were synthesized in essentially quantitative yields and high purities via mechanochemical salification, and one novel organic HCl salt was produced via slow evaporation, as evidenced by PXRD and ³⁵Cl SSNMR. Optimized LAG conditions and ball milling parameters are reported in FIG. 24. Generally, the role of ball milling appears to be that of mixing of reagents, consistent with the acid-base neutralization reactions that often occur spontaneously for such systems. However, there are many factors that play a role in the formation of the desired salt with minimal impurities and waste, as discussed in the text that follows.

Impact of the Quantity of HCl. In most solution-based salification reactions, HCl is added in significant excess (e.g., fully dissolving a solid free base in concentrated HCl);^{7,3} however, with mechanochemical methods, it is possible to use microliters of solvent in precise molar ratios to target specific products. Generally, if multiple solid phases differ in their chloride content, the relative stoichiometry of HCl governs which phase is obtained. For example, NicH-Nic:H₂O and NicH were both synthesized via LAG using precise stoichiometric amounts of 12.1 M HCl(aq) to target the desired product (i.e., half an equivalent was used for NicH-Nic:H₂O, whereas one equivalent was used for NicH). If more than half an equivalent of HCl is added, the formation of NicH is favored due to preferred protonation of the pyridyl nitrogen. A slight excess of acid can also facilitate the synthesis of a specific product by preventing side reactions or driving the reaction forward in cases where optimizing other synthetic parameters is unfeasible (e.g., in the salification reactions that yield AcetH:H₂O and PicAH, two equivalents of HCl is used to eliminate impurities and/or unreacted educts).

Impact of the Solvent of HCl Solutions. The choice of solvent can have a considerable impact on mechanochemical reactions.^{31,113-115} Herein, three distinct solvents were used for concentrated HCl solutions, including 12.1 M HCl(aq), 3.0 M methanolic HCl (HCl(MeOH)) and 1.25 M ethanolic HCl (HCl(EtOH)). In the cases of PicA, Caff, and Acet, the solvent determines which salt is formed. Use of HCl(aq) leads to the production of the hydrates PicAH:H₂O, CaffH:H₂O, and AcetH:H₂O, whereas use of HCl(MeOH) or HCl(EtOH) leads to the formation of the anhydrous salts PicAH, CaffH, and AcetH. This contrasts with the case of NicA, where the solvent is relatively unimportant, as NicAH appears to be the only possible product (i.e., no hydrate or solvate phases have been observed in our reaction product mixtures). The use of different HCl solvents also offers insights into the nature of the products. For example, PicAH-PicA was determined to be anhydrous due to the identical products obtained from reactions involving either HCl(aq) and HCl(MeOH). Similarly, IsoH-Iso:H₂O was determined to be a hydrate, since use of half an equivalent of HCl(MeOH) produced a mixture of IsoH and unreacted Iso.

Hence, altering both the amount of HCl and its solvent offers additional pathways for synthesizing novel materials. In certain cases, different HCl solvents may result in different reaction mechanisms that either lead to the same product, or perhaps a distinct polymorph or solvate.^{25,31,55,116-119} As such, it is possible that other systems may exhibit even more complex behavior. However, use of HCl(aq) is generally preferred, since its high HCl concentration is associated with significantly less solvent in comparison to concentrated alcoholic HCl (e.g., in the preparation of 100 mg of NicH, 70 μL of HCl(aq) is necessary, which corresponds to ca. 300 μL of HCl(MeOH) or 700 μL of HCl(EtOH)). However, methanol and ethanol are more easily evaporated than water, given that they are ca. five and two times as volatile under standard temperature and pressure (this is based on calculations using the Clausius-Clapeyron equation, assuming boiling points of 64.7, 78.4, and 100.0° C. for MeOH, EtOH, and H₂O, respectively).

Impact of Milling Duration and Milling Frequency. Two important considerations for almost all ball milling reactions are the milling duration and milling frequency. Generally, a sufficient milling duration is required to drive the reaction to completion and ensure formation of the desired products, whereas the milling frequency impacts the rate of the reaction. In most of the cases presented herein, milling at 20 Hz for 5 to 15 minutes is sufficient for the formation of an HCl salt with no trace of educts (i.e., unreacted free bases) or other impurities. However, as the milling duration or milling frequency increases, the temperature inside the milling jar increases as well, which may impact the reaction and end products.¹²⁰

Heating hydrates of organic HCl salts sometimes results in the release of water, leading to an anhydrous solid. For example, excess heating of CaffH:H₂O causes dehydration and disproportionation to Caff, releasing H₂O and HCl.¹²¹ This effect can be replicated in LAG reactions intended for the production of CaffH:H₂O, where milling at a higher frequencies and/or longer durations results in the presence of Caff impurities.

The synthetic pathways for the various HCl salts of PicA (FIG. 1) highlight the heat-dependent transformation of PicAH-H₂O to PicAH-PicA. If the desired product is PicAH:H₂O, the formation of PicAH-PicA needs to be prevented. There are several options to accomplish this, all of which can be used in tandem: (i) addition of excess HCl(aq) causes any PicAH-PicA that is formed to convert to PicAH:H₂O; (ii) use of cryogens can be employed to avert the buildup of heat, preventing the formation of PicAH-PicA; and (iii) reducing the milling time and/or frequency can be used to decrease the time the material is exposed to heat and/or reduce the rate of heat generation. Other parameters also impact the maximum temperature reached, such as the materials of the jar and ball bearings, as well as the amount of material being milled. These are very important considerations for scaling up the reaction size for industrial production.¹²⁰

Impact of pKₐ and Material Stability—An Investigation Using Competitive Milling. The protonation behavior of the molecules is strongly influenced by their functional groups and positions on the pyridyl ring. Amide groups, such as those in Nic, Iso, and Pic, act as weak electron-withdrawing substituents, which decreases the basicity of the pyridyl nitrogen and makes protonation less favorable in comparison to an unsubstituted pyridyl ring (e.g., pKₐ's of the pyridyl nitrogen are 3.4 and 5.2 for Nic and pyridine, respectively). Similarly, carboxylic acid moieties, such as those in NicA, IsoA, and PicA, serve as stronger electron-withdrawing groups, further reducing the basicity of the pyridyl nitrogen and making protonation even less favorable. The position of the substituent relative to the pyridyl nitrogen also plays a key role in modulating electronic effects. Electron-withdrawing groups positioned closer to the pyridyl nitrogen exert a stronger inductive effect, as in the case of Pic, where electron density is withdrawn more effectively from the pyridyl nitrogen than in Nic or Iso. Furthermore, substituents in the ortho and para positions engage in resonance with the pyridyl ring, resulting in a greater reduction in basicity than would be predicted from inductive effects alone.

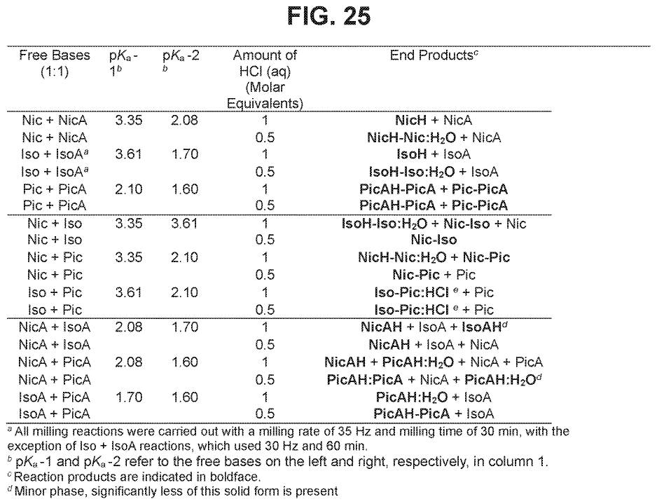

To investigate the impact of these effects in the context of mechanochemical salification, we conducted a series of competitive milling reactions (FIG. 25).^{122,123} An equimolar amount of two free bases (e.g., Nic and NicA) were added to a milling jar with either one or half a molar equivalent of HCl(aq), and ball milled. The product was then characterized to determine which phase(s) of material is(are) formed.

The enthalpies of formation (ΔH_{f}) were derived from the equation:

$$Δ {H}_{f} = {H}_{product} - ( {AH}_{educt}^{( 1 )} + {BH}_{educt}^{( 2 )} + {CH}_{water} )$$

where H_{product}, H_{educt}, and H_{water} correspond to the DFT-derived static lattice energies of the products, educts, and water, respectively, and the coefficients A, B, and C correspond to the stoichiometric amounts of each educt and water, where H_{water} is only considered for hydrates. While many of the values of AHI are only slightly negative or even positive, there are several possible explanations for why the reaction still proceeds: (i) some portion of the solid is dissolved, which is not captured in static lattice energy models;²¹ (ii) the reaction may be driven through product precipitation, which shifts the equilibrium irreversibly forward;³¹ (iii) there are relatively large errors in these calculated values as finite temperature effects are not taken into account;¹²⁵⁻¹²⁷ and (iv) mechanochemical effects, such as defect formation and local heating, contribute additional energy to drive the reaction forward.^{128,129}

First, the competitive milling reactions of mixtures of amides and carboxylic acids with HCl are considered. For Nic, NicA, Iso, and IsoA, the reactions proceed as expected, where the more basic Nic and Iso molecules are protonated instead of NicA and IsoA (i.e., only NicH and IsoH are formed, perhaps due to the higher pKₐ's of their pyridyl nitrogens). However, similar reactions with Pic and PicA yield the salt PicAH-PicA, as well as a picolinamide picolinic acid cocrystal (Pic-PicA),¹³⁰ meaning that Pic reacts with the weak acid PicA, rather than the significantly stronger HCl. Without a crystal structure for Pic-PicA, it is difficult to say whether this is a result of a kinetically-favored reaction or if the cocrystal is more thermodynamically stable than PicH, PicH:H₂O, PicAH, and/or PicAH:H₂O.

Second, reactions of mixtures of amides with HCl are discussed. Both the 1:1:1 and 1:1:0.5 reactions of Nic, Iso, and HCl yield a nicotinamide-isonicotinamide cocrystal (Nic-Iso).¹³⁰ This is interesting, as the ΔH₁ for Nic-Iso is significantly higher than that of NicH or IsoH, meaning a less thermodynamically favorable product is formed. We observe the salt IsoH-Iso:H₂O and excess Nic in the 1:1:1 case, suggesting that excess acid is needed to drive the reaction forward. Similarly, reactions of Nic, Pic, and HCl yield NicH-Nic:H₂O as well as a nicotinamide-picolinamide cocrystal (Nic-Pic),¹³⁰ further highlighting the possibility of kinetically driven reactions. We also note that NicH-Nic:H₂O is formed in the 1:1:1 reaction, suggesting that Nic-Pic formation competes poorly under these conditions in comparison to Nic-Iso and Pic-PicA. The reactions of Iso, Pic, and HCl result in the production of an unknown solid form, which is suspected to be an ionic cocrystal of Iso, Pic, and HCl (Iso-Pic:HCl) as evidenced by a unique ³⁵Cl SSNMR spectrum, but this is not explored further in this work. However, since there is PXRD evidence of unreacted Pic in the final reaction mixture, it is likely that this cocrystal does not have a 1:1:1 stoichiometric ratio of the three components.

Finally, the reactions of mixtures of carboxylic acids with HCl are explored. NicA, IsoA, and HCl react in a 1:1:1 ratio to form NicAH and IsoA; however, some IsoAH is also observed, whereas the 1:1:0.5 reaction yields NicAH as the only salt, with excess IsoA and NicA. However, the 1:1:1 NicA+PicA+HCl reaction yields NicAH, PicAH:H₂O, and educts, whereas the 1:1:0.5 reaction yields PicAH-PicA, PicAH:H₂O, and residual NicA. Both reaction ratios for IsoA, PicA, and HCl yield PicAH:H₂O as the only salt (plus residual IsoA).

In these competitive milling reactions, neither the pyridyl nitrogen basicity (as predicted by pKₐ) nor thermal stabilities are the sole predictors of the products. Even the assumption that one of these educts would react with HCl is sometimes incorrect, as seen by the formation of the Pic-PicA, Nic-Iso, and Nic-Pic cocrystals. In mechanochemical reactions, there are additional factors to consider. First, there are clear limitations of using pKₐ values to predict the outcomes of solid-state reactions, since they are derived from solution measurements, and do not account for the array of additional solid-state interactions, including intermolecular hydrogen bonding and molecular packing that influence intrinsic basicity/acidity. Second, since mechanochemical reactions involving organic compounds are thought to initiate on the surfaces of particles that are being ground or sheared together,¹³¹⁻¹³³ it is possible that some reactions are kinetically driven, which may supplant simple considerations of acidity, basicity, and/or thermodynamics. While we are unable to provide a succinct and satisfying explanation for these phenomena, it is clear that predictions based on simple solution-phase principles do not apply to mechanochemical reactions; nonetheless, this opens a wider canvas for exploration of the production of HCl salts and related cocrystals.

### Mechanochemical Screening of Novel Phases of Hemihydrochloride Salts

High-throughput recrystallizations are often preferred for screening due to the relative ease of setting up many reactions under a variety of synthetic conditions.^{134,135} However, mechanochemistry may have an edge in discovering more unusual phases in situations where the stoichiometric ratio of the organic free base (FB) to the number of chloride ions is greater than 1:1, such as in the case of hemihydrochloride salts (e.g., FB:HCl=1:0.5, like in the cases of IsoH-Iso:H₂O, PicAH-PicA, etc.). For example, NicH-Nic:H₂O was first synthesized via a two-step slow evaporation over the course of several weeks.⁷¹ However, we report its quantitative synthesis in as little as 2 min, with no detectable byproducts or impurities. The ability to target specific molar ratios with HCl in various solvents allows for facile screening using mechanochemical methods and the exploration of novel solid forms.

### Syntheses of Novel HCl Salts

Here, we discuss the mechanochemical syntheses of five novel materials: IsoH-Iso:H₂O, PicH-Pic, PicH, PicAH-PicA, AcetH-Acet:H₂O, along with the formation of PicH:H₂O via slow evaporation (reaction summaries are again given in FIG. 24). The hemihydrochloride salts (i.e., IsoH-Iso:H₂O, PicH-Pic, PicAH-PicA) were synthesized with ratios of 1:0.5 FB:HCl. We also attempted to prepare these materials with 12.1 M HCl(aq) or 3.0 M HCl(MeOH) to confirm their hydration states, finding that either solvent led to the formation of PicH-Pic and PicAH-PicA, whereas only HCl(aq) led to IsoH-Iso:H₂O. PicH can be formed with a 1:1 ratio of FB:HCl with either HCl(EtOH) or HCl(MeOH), suggesting that it is anhydrous. Ball milling Acet and 12.1 M HCl(aq) in a 1:1 molar ratio yields what we suspect to be AcetH-Acet:H₂O, since a 1:2 ratio yields AcetH:H₂O and a 1:0.5 ratio yields a mixture of Acet and AcetH-Acet:H₂O. Due to the relatively poor basicity of the secondary amide, it is unclear whether AcetH-Acet:H₂O is a hemihydrochloride requiring optimized milling conditions, if it is a polymorph of AcetH:H₂O, or if it exists in a unique, non-standard molar ratio. While mechanochemical methods alone cannot give a definitive answer on the nature of this material, TGA can.

The synthesis of PicH:H₂O is unique in this work, being non-mechanochemical. Two equivalents of HCl(aq) are enough to fully dissolve Pic while milling, with subsequent evaporation of excess solvent leading to the formation of PicH:H₂O. The formation of this material is likely better suited for slow evaporation, as straightforward milling of PicH with different molar equivalents of H₂O does not lead to the formation of PicH:H₂O. This slow evaporation synthesis yielded crystals suitable for SCXRD structure determination.

### Mechanochemical Scale-Up

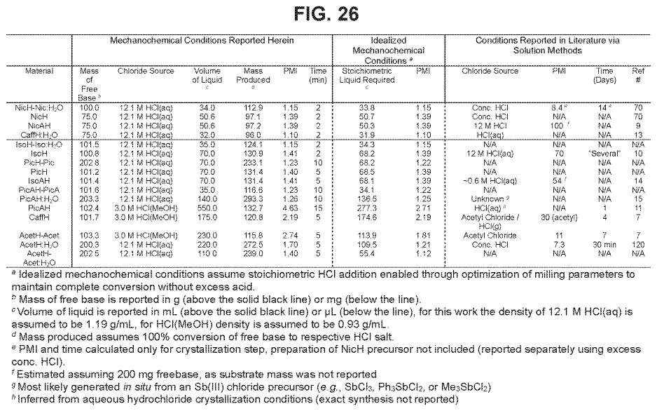

The mechanochemical salification methods outlined herein have the potential to be used on an industrial scale. To explore this, a speed mixer was used with larger quantities of free base solids and 12.1 M HCl(aq) to produce NicH, NicH-Nic:H₂O, NicAH, and CaffH in amounts between 75 and 100 g in quantitative yields (i.e., no detectable educts or impurities in the PXRD patterns) in as little as 2 minutes at 2000 RPM (FIG. 26, first four rows). This was accomplished using the same reaction chemistries listed in FIG. 24, with near stoichiometric amounts of HCl and no ball bearings. Further reactions to even larger scales were not attempted due to weight limitations of the speed mixer in our laboratory; however, this shows promise for scaling such reactions into the kilogram regime with the appropriate equipment. As these salification reactions are exothermic (i.e., reaction vessels became warm to the touch), thermal effects should be considered during further scale-up, particularly when working with concentrated HCl and larger batch masses under confined mixing conditions. Again, of particular note are the high yields, minimal impurities, low solvent use, and reduced time scales compared to solvent-based recrystallizations. These reactions also maintain low process mass intensities (PMIs) at larger scales (FIG. 26), reflecting the near-stoichiometric use of HCl and minimal liquid loading relative to conventional hydrochloride salt crystallization methods.

### Structural Characterization of Organic HCl Salts

Single-crystal X-ray Diffraction. Slow evaporations were attempted for all novel materials in hopes of obtaining crystals suitable for analysis by SCXRD; however, only recrystallizations of PicH:H₂O were successful. PicH:H₂O crystallizes in the monoclinic space group P2₁/n featuring a single chloride ion site. This crystal structure is available from the Cambridge Structural Database under deposition number 2341820.

Thermal Analysis. TGA was used to characterize the novel hemihydrochloride salts IsoH-Iso:H₂O, PicH-Pic, PicAH-PicA, and AcetH-Acet:H₂O, with the goal of determining their hydration state. First, since the molar mass of IsoH-Iso:H₂O is 298.73 g mol-1, ca. 6% of its mass is attributed to water. This is confirmed by a ca. 7% mass reduction over a temperature range from 35 to 145° C. (thermal degradation occurs at ca. 155° C. The additional ca. 1% loss may arise from moisture content in the sample. Second, the TGA results for PicH-Pic and PicAH-PicA are inconclusive due to the inherent thermal instability of their components. PicAH thermally degrades at temperatures as low as 55.2° C., and Pic has an even lower melting point than PicA (i.e., 110° C. vs. 136° C.), making both materials poor candidates for thermal analysis.¹³⁶ Consistent with this, thermal degradation for PicH-Pic and PicAH-PicA begins near 80° C. Prior to this, there is a small mass reduction of ca. 5%, which could arise from either the partial release of waters of hydration, resulting in a smaller than expected weight loss for a monohydrate, or the presence of “surface” water molecules (i.e., physisorbed, chemisorbed, and/or interstitial water molecules that are not part of the crystalline lattice). However, these materials can be synthesized under anhydrous conditions, making the presence of waters of hydration unlikely. Finally, AcetH-Acet:H₂O, with a molecular mass of 356.806 g mol-1 and anticipated mass reductions of 15.3% and 19.3% for mono- and di-hydrate forms, respectively, has an experimental reduction of ca. 17% over a temperature range from 65° C. to 175° C., before decomposing at ca. 210° C. As such, AcetH-Acet:H₂O is likely a monohydrate and the remainder of the mass loss (~2.7%) is due to the volatilization of surface water molecules. It is also possible that AcetH-Acet:H₂O contains a non-stoichiometric amount of water, such as 1.5 water molecules per asymmetric unit, which would result in a 17.4% mass loss.⁵³

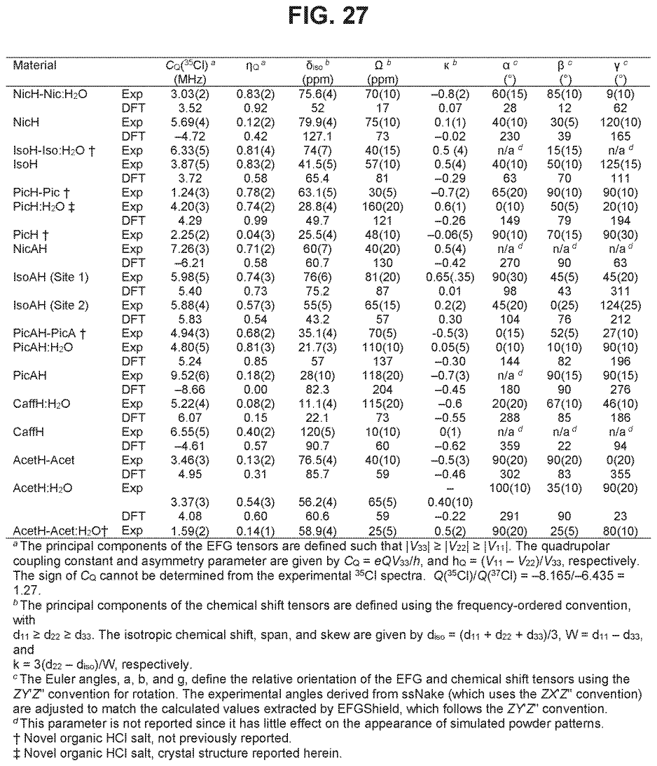

^{35/37}Cl SSNMR. ³⁵Cl SSNMR spectra were acquired for all HCl salts under static conditions at 18.8 T, as well as under MAS conditions at 18.8 T for most samples. In order to accurately determine the EFG and CS tensor parameters, ³⁵Cl SSNMR spectra are usually acquired at two different fields. However, since chlorine has a second NMR-active isotope, 37Cl (l=3/2), 37Cl SSNMR spectra were acquired for most HCl salts under static conditions at 18.8 T to enable a similar analysis.^{48,49,65} MAS experiments, which can average the contributions from CSA, but only partially average those from the SOQI, enable the acquisition of spectra that allow for the facile determination of C_{Q}, η_{Q}, and δᵢₛₒ. In turn, this facilitates simulations of corresponding static NMR spectra (the latter require eight parameters to fit, including the CS tensors and Euler angles that describe the relative orientation of the EFG and CS tensors;¹³⁷ see FIG. 27 for definitions).

35/37Cl SSNMR spectra for each form feature CT patterns that are different from one another, demonstrating the utility of^{35/37}Cl SSNMR for spectral fingerprinting. The magnitudes of Ca reflect the degree of spherical symmetry of the ground state electron density about the Cl— ion (increased magnitudes indicate less spherical symmetry), while the η_{Q} values describe the axial asymmetry of the EFG tensor (0≤η_{Q}≤1, where η_{Q}=0 is axial). All of this, combined with the relatively short acquisition times for these spectra, makes ^{35/37}Cl SSNMR an ideal technique for not only obtaining spectral fingerprints, but also for studying the relationships between the ^{35/37}Cl EFG tensors and hydrogen bonding environments of the chloride ions.

The appearances of the CT patterns are dominated by the SOQI, having breadths ranging from 10 to 150 kHz. All spectra of the known HCl salts feature a single CT pattern corresponding to single distinct chloride site, with the exception of those of IsoAH, which feature two overlapping powder patterns, in agreement with its crystal structure.¹³⁸ The spectra show no evidence of unwanted side products (i.e., other organic HCl salts or sharp peaks indicating hydrated chloride ions), indicating that the reaction conditions for salt formation are selective for specific products.

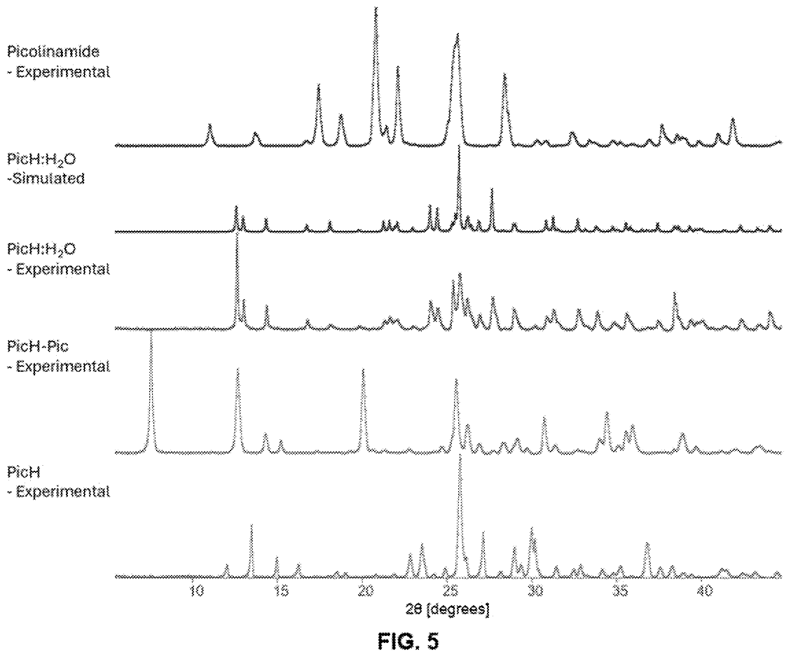

We now consider the ³⁵Cl SSNMR spectra from the perspectives of phase identification and structural interpretation, first discussing the HCl salts of Nic, Iso, NicA, and IsoA (FIG. 5A). NicH and NicH-Nic:H₂O have distinct ³⁵Cl CT powder patterns, with the former arising from a moderate C_{Q} and low η_{Q}, and the latter from a small C_{Q} and high η_{Q}. IsoH and IsoH-Iso:H₂O are also readily differentiable, as the C_{Q} for IsoH is much smaller, and both EFG tensors are non-axially symmetric. The ³⁵Cl CT patterns of NicAH and IsoAH are different from those of NicH and IsoH, with a large C_{Q} and non-axial η_{Q} for NicAH, and two individual patterns of with moderate C_{Q}'s and variable η_{Q}'s for IsoAH (vide supra). Second, the HCl salts of Pic and PicA have very different ³⁵Cl CT patterns from simple salts, hydrates, and hemichlorides, with the exception of those of PicAH-PicA and PicAH:H₂O, which have powder patterns yielding similar quadrupolar parameters but different values of δᵢₛₒ. Finally, ³⁵Cl CT patterns for CaffH and CaffH:H₂O, as well as AcetH:H₂O, AcetH-Acet, and AcetH-Acet:H₂O, all have unique shapes and distinct sets of quadrupolar and chemical shift parameters. From this dataset, and a broader review of the data that follows, there are no clear trends in C_{Q} and η_{Q} that can be directly related to the general form of the solid (e.g., simple salts do not necessarily have chloride ions with smaller C_{Q} values or low η_{Q} values in comparison to their corresponding hydrated forms); rather, differences arise due to the nature of the local Cl⁻ hydrogen bonding environments (vide infra).

³⁵Cl EFG tensors and Molecular-Level Structure

Here, we compare the ³⁵Cl EFG tensors derived from quantum chemical computations with those obtained experimentally and consider their relationships with the local structural environment of the chloride ions. Dispersion-corrected plane-wave DFT geometry optimizations ^{101,102,139} were conducted on structural models based on their respective crystal structures; subsequently, ³⁵Cl EFG tensors were calculated (FIG. 27). A comparison of experimental and computed EFG tensors shows a reasonable correlation, with an RMS EFG distance (Γ_{EFG}) of 0.66 MHz.^{62,102} However, significant outliers are observed for AcetH-Acet, CaffH, and CaffH:H₂O, possibly due to dynamics that partially average their quadrupolar interactions that are not reflected in the DFT calculations, which are conducted on static structural models. To further investigate this, ³⁵Cl spectra of CaffH:H₂O and AcetH-Acet were acquired at 245 K. In both cases, there is an increase in the magnitudes of both C_{Q} and η_{Q} at the lower temperature, bringing experimental and calculated values into better agreement. Removing these outliers from the original correlation plot results in better agreement, with a Γ_{EFG}=0.51 MHz.

The ³⁵Cl EFG tensors of chloride ions depend on the surrounding H . . . Cl⁻ bonds, including their number, their lengths [r(H . . . Cl⁻)], the types of moieties involved in bonding, and their spatial arrangement. Some information on H . . . Cl⁻ bonds can be garnered directly from the quadrupolar parameters; however, DFT calculations are useful for closely examining these relationships. We note that typical H . . . Cl⁻ bonds are classified as having r(H . . . Cl⁻)≤2.6 Å; ¹⁴⁰ however, it is the short H . . . Cl⁻ contacts, i.e., r(H . . . Cl⁻)≲2.2 Å, which largely dictate the properties of the ³⁵Cl EFG tensors.^{58,61}

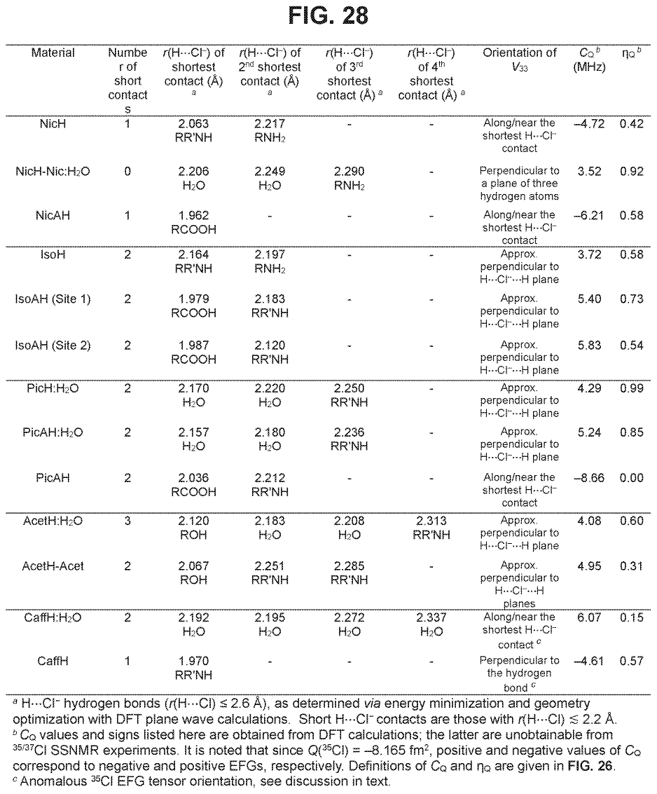

Summarizing, the ³⁵Cl EFG tensor orientations, magnitudes and signs of C_{Q}, and structural relationships are consistent with those from previous reports on a wide range of organic HCl salts (FIG. 28). For systems with one short H . . . Cl⁻ or two short H . . . Cl⁻ contacts with ∠(H . . . Cl . . . H)≥140°, the magnitudes of C_{Q} are moderate to large, their signs are negative (i.e., positive V₃₃ components), and V₃₃ is oriented along or near the direction of the shortest contact. In cases with two short contacts, small to moderate magnitudes of C_{Q} are observed, their signs are positive, with V₃₃ oriented approximately perpendicular to the H . . . Cl⁻. . . H planes. Values of r(H . . . Cl⁻) are generally found to be shorter for short contacts involving RCOOH moieties than those associated with 1° and 2° amides. The situation becomes more complex for two or three short contacts and additional hydrogen bonds—this is where DFT calculations have great value. Below, we discuss the ³⁵Cl EFG tensors and their orientation and the impact of different H . . . Cl⁻ contacts, including r(H . . . Cl⁻) and the identities of the hydrogen-bonding moieties (FIG. 28).

We begin by comparing NicAH, NicH, and NicH-Nic:H₂O. NicAH and NicH each have a single short contact, which results in moderate values of Ca (−6.21 MHz and −4.72 MHz, respectively), with V₃₃, aligning near the axis of the short contact. The magnitude of C_{Q} is larger for NicAH due to the RCOOH moiety, which generally has shorter r(H . . . Cl⁻) values than amide groups, and hence, larger magnitudes of C_{Q}. NicH-Nic:H₂O is distinct, with three H . . . Cl⁻ bonds in a pyramidal arrangement, leading to a C_{Q} with a relatively small magnitude (3.52 MHz) and sign opposite to those above, with V₃₃ oriented roughly perpendicular to a plane formed by the three hydrogen bonds.

IsoH and IsoAH have one and two chloride ion sites, respectively. The chloride ion environments all feature two short contacts arranged in “V” formations. The Ca values are all positive, with larger magnitudes in IsoAH because of the RCOOH moieties, which result in shorter values of r(H . . . Cl⁻). In all cases, V₃₃ is oriented approximately perpendicular to the H . . . Cl⁻. . . H plane.

We next compare PicH:H₂O, PicAH:H₂O, PicAH, and PicH. For the hydrates PicH:H₂O and PicAH:H₂O, the Ca values are positive, with V₃₃ oriented perpendicular to the H . . . Cl⁻. . . H planes featuring an RR′NH moiety and H₂O molecule. PicAH has a large C_{Q}=−8.66 MHz and η_{Q}=0.0, which results from two short H . . . Cl⁻ contacts with hydrogen atoms positioned on opposite sides of the chloride ion,^{58,122} and V₃₃ aligned closely to the shorter H . . . Cl⁻ contact. This stands in contrast with PicH (for which the crystal structure is unknown), which has a small experimentally-determined C_{Q}=2.25 MHz, suggesting the presence of longer hydrogen bonds with surrounding 1° and 2° amides).

AcetH:H₂O and AcetH-Acet both have low-to-moderate C_{Q} values. AcetH-Acet has three short H . . . Cl⁻ contacts and a low rna value. The C_{Q} magnitude is due to the comparatively larger number of contacts, whereas the latter results from the pyramidal arrangement of hydrogen bonds that approaches C₃ symmetry. AcetH:H₂O has two short contacts and two additional hydrogen bonds with H₂O molecules. In both cases, C_{Q} is positive and the alignment of V₃₃ is roughly perpendicular to the short contacts.

Finally, CaffH and CaffH:H₂O have distinct chloride ion environments. CaffH has a single short contact (RR′NH), and as expected, a negative, moderate C_{Q}. However, V₃₃ is oriented perpendicular to this bond rather than along it, which is unusual. CaffH:H₂O has four hydrogen bonds, two of which are short contacts, all arising from water molecules, which is a coordination motif rarely observed in organic HCl salts. V₃₃ is oriented near the shortest hydrogen bond. The uncommon hydrogen bonding networks in CaffH and CaffH:H₂O may account for the anomalous ³⁵Cl EFG tensor orientations and contribute to the discrepancies between calculated and experimental NMR parameters; for CaffH:H₂O, this may also be the result of dynamical motions of the water molecules that influence the experimentally measured ³⁸Cl EFGs.

### CONCLUSIONS

This study demonstrated the (i) rapid and quantitative mechanochemical syntheses of sixteen HCl salts, including anhydrous forms, hydrates, and hemihydrochlorides (five of which are novel), as well as the formation of PicH:H₂O via slow evaporation; (ii) advantages of mechanochemical salification, including negligible waste, minimal solvent use, quantitative yields, accelerated discovery of solid forms, and scalability; (iii) complexities inherent to solid-state chemistry, where common assumptions and behaviors derived from solution chemistry do not apply (e.g., a myriad of factors can influence kinetic vs. thermodynamic products and the limitations of using pKₐ's to make predictions regarding protonation of pyridyl nitrogens; (iv) value of ^{35/37}Cl SSNMR spectroscopy for fingerprinting of chloride salts, impurity detection, and structural elucidation; and (v) the application of plane-wave DFT calculations of ³⁵Cl EFG tensors to investigate their relationships to the hydrogen bonding environments of chloride ions.

The combination of mechanochemical salification and characterization via ^{35/37}Cl SSNMR offers substantial benefits at both laboratory and industrial scales. On the laboratory side of things, screening for solid forms is significantly accelerated, making the discovery and identification of rare solid forms, such as hemihydrochlorides, more straightforward. At an industrial scale, mechanochemistry has great potential for increased efficiency and reduced costs, which benefits the manufacturer and consumer, due to reduction in solvent, energy use, safety hazards, waste disposal, and unwanted by-products. The usefulness of ^{35/37}Cl SSNMR in laboratory settings is clear from this work and many preceding accounts; however, it could play an important role in industrial settings for spectral fingerprinting and impurity detection, especially since MAS experiments are not absolutely required (this eliminates the costs of rotors, tedious sample packing, and opens the possibility of looking at complex dosage formulations like pills and capsules directly)—and, only a single magnetic field is necessary.^{57,141} Furthermore, it could aid in the quantification of HCl salts, examination of disproportion,¹⁴² and streamlining of early-stage screening and selection, all of which are essential for time-sensitive discovery and in formulation pipelines, where rapid decisions on salt forms are needed prior to scaling up or advancing through regulatory pathways.

While the current work focuses specifically on organic HCl salts, this mechanochemical salification method is broadly applicable to a wider range of APIs. The same principles could be extended to form salts with polyprotic acids (e.g., H₂SO₄, H₃PO₄, etc.) or other pharmaceutically relevant counterions (e.g., HBr, HNO₃, methanesulfonic acid, etc.), potentially enabling the rapid generation of diverse salt forms beyond those traditionally accessed via solution-based methods. This versatility highlights the utility of mechanochemistry for the development of many new solid forms across a vast landscape of APIs.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

(1) Hamaed, H.; Pawlowski, J. M.; Cooper, B. F. T. T.; Fu, R.; Eichhorn, S. H.; Schurko, R. W. Application of Solid-State 35Cl NMR to the Structural Characterization of Hydrochloride Pharmaceuticals and Their Polymorphs. J Am Chem Soc 2008, 130, 11056-11065. https://doi.org/10.1021/ja802486q.
(2) Paulekuhn, G. S.; Dressman, J. B.; Saal, C. Trends in Active Pharmaceutical Ingredient Salt Selection Based on Analysis of the Orange Book Database. J Med Chem 2007, 50, 6665-6672. https://doi.org/10.1021/JM701032Y.
(3) Muleva, C. T.; Bharate, S. S. Halide Counterions in FDA-Approved Pharmaceutical Salts. J Drug Deliv Sci Technol 2023, 89, 104999. https://doi.org/10.1016/J.JDDST.2023.104999.
(4) Bharate, S. S. Recent Developments in Pharmaceutical Salts: FDA Approvals from 2015 to 2019. Drug Discov Today 2021, 26, 384-398. https://doi.org/10.1016/J.DRUDIS.2020.11.016.
(5) Hossain Mithu, M. S.; Economidou, S.; Trivedi, V.; Bhatt, S.; Douroumis, D. Advanced Methodologies for Pharmaceutical Salt Synthesis. Cryst Growth Des 2021, 21, 1358-1374. https://doi.org/https://doi.org/10.1021/acs.cgd.0c01427.
(6) Noble, E. G.; Shanks, D. E. Hydrogen Chloride and Hydrogen Bromide Sparging Crystallization of Chloride and Bromide Metal Salts. Crystallization and Precipitation 1987, 89-98. https://doi.org/10.1016/B978-0-08-035751-5.50019-2.
(7) Perumalla, S. R.; Sun, C. C. Confused HCl: Hydrogen Chloride or Hydrochloric Acid?Chemistry—A European Journal 2012, 18, 6462-6464. https://doi.org/10.1002/CHEM.201103669.
(8) Perumalla, S. R.; Shi, L.; Sun, C. C. Ionized Form of Acetaminophen with Improved Compaction Properties. CrystEngComm 2012, 14, 2389-2390. https://doi.org/10.1039/C1CE06278F.
(9) Slouf, M. 3-Carboxypyridinium Chloride. Acta Crystallogr Sect E Struct Rep Online 2001, 57, o61-o62. https://doi.org/10.1107/S1600536800019632.
(10) Fellows, S. M.; Prior, T. J. Crystal Structure of 4-Carbamoylpyridinium Chloride. Acta Crystallogr E Crystallogr Commun 2016, 72, 436-439. https://doi.org/10.1107/S2056989016003340.
(11) Laurent, A.; IUCr. Structure Cristalline Du Chlorhydrate de l'acide Picolique. Acta Cryst. 1965, 18, 799-806. https://doi.org/10.1107/S0365110X65001809.
(12) Gubin, A.; Nurakhmetov, N.; Buranbaev, M.; Mulkina, R.; Erkasov, R. Crystal and Molecular-Structure of Nicotinamide Hydrochloride—Donor-Acceptor Properties of Protonated Nicotinamide. Kristallografiya 1989, 238-239.
(13) Mercer, A.; Trotter, J.; IUCr. Crystal and Molecular Structure of 1,3,7-Trimethyl-2,6-Purinedione Hydrochloride Dihydrate (Caffeine Hydrochloride Dihydrate). Acta Cryst. 1978, 34, 450-453. https://doi.org/10.1107/S0567740878003337.
(14) Adams, C. J.; Angeloni, A.; Orpen, A. G.; Podesta, T. J.; Shore, B. Crystal Synthesis of Organic-Inorganic Hybrid Salts Based on Tetrachloroplatinate and -Palladate Salts of Organic Cations: Formation of Linear, Two-, and Three-Dimensional NH . . . Cl Hydrogen Bond Networks. Cryst Growth Des 2006, 6, 411-422. https://doi.org/https://doi.org/i0.1021/cg050362o.
(15) Smith, G.; Byriel, K. A.; Kennard, C. H. L.; IUCr. 2-Carboxypyridinium Chloride Monohydrate. Acta Cryst. 1995, C51, 1425-1427. https://doi.org/10.1107/SO108270195000175.
(16) Nakagawa, K.; Murakami, W.; Andrieu, J.; Vessot, S. Freezing Step Controls the Mannitol Phase Composition Heterogeneity. Chemical Engineering Research and Design 2009, 87, 1017-1027. https://doi.org/10.1016/J.CHERD.2009.01.008.
(17) Lee, S.; Wi, H. S.; Jo, W.; Cho, Y. C.; Lee, H. H.; Jeong, S. Y.; Kim, Y. II; Lee, G. W. Multiple Pathways of Crystal Nucleation in an Extremely Supersaturated Aqueous Potassium Dihydrogen Phosphate (KDP) Solution Droplet. Proc Natl Acad Sci USA 2016, 113, 13618-13623. https://doi.org/doi.org/10.1073/pnas.1604938113.
(18) Mejri, E.; Bouhlila, R.; Helmig, R. Heterogeneity Effects on Evaporation-Induced Halite and Gypsum Co-Precipitation in Porous Media. Transp Porous Media 2017, 118, 39-64. https://doi.org/doi.org/10.1007/s11242-017-0846-8.
(19) Pagola, S. Outstanding Advantages, Current Drawbacks, and Significant Recent Developments in Mechanochemistry: A Perspective View. Crystals (Basel) 2023, 13, 124. https://doi.org/i0.3390/CRYST13010124.
(20) Friščić, T.; Mottillo, C.; Titi, H. M. Mechanochemistry for Synthesis. Angewandte Chemie—International Edition 2019, 1018-1029. https://doi.org/i0.1002/anie.201906755.
(21) Do, J.-L.; Friščić, T. Mechanochemistry: A Force of Synthesis. ACS Cent Sci 2017, 3, 13-19. https://doi.org/10.1021/acscentsci.6b00277.
(22) Crawford, D. E.; Miskimmin, C. K. G.; Albadarin, A. B.; Walker, G.; James, S. L. Organic Synthesis by Twin Screw Extrusion (TSE): Continuous, Scalable and Solvent-Free. Green Chemistry2017, 19, 1507-1518. https://doi.org/10.1039/C6GC03413F.
(23) Rensch, T.; Chantrain, V.; Sander, M.; Gretz, S.; Borchardt, L. Scale-Up of Solvent-Free, Mechanochemical Precursor Synthesis for Nanoporous Carbon Materials via Extrusion. ChemSusChem 2022, 15, e202200651. https://doi.org/i0.1002/CSSC.202200651.
(24) Stolar, T.; Lukin, S.; Tireli, M.; Sović, I.; Karadeniz, B.; Kereković, I.; Matijašić, G.; Gretić, M.; Katančić, Z.; Dejanović, I.; Michiel, M. Di; Halasz, I.; Užarević, K. Control of Pharmaceutical Cocrystal Polymorphism on Various Scales by Mechanochemistry: Transfer from the Laboratory Batch to the Large-Scale Extrusion Processing. ACS Sustain Chem Eng 2019, 7, 7102-7110. https://doi.org/i0.1021/acssuschemeng.9b00043.
(25) Stolle, A.; Schmidt, R.; Jacob, K. Scale-up of Organic Reactions in Ball Mills: Process Intensification with Regard to Energy Efficiency and Economy of Scale. Faraday Discuss 2014, 170, 267-286. https://doi.org/10.1039/C3FD00144J.
(26) Madanayake, S. N.; Manipura, A.; Thakuria, R.; Adassooriya, N. M. Opportunities and Challenges in Mechanochemical Cocrystallization toward Scaled-Up Pharmaceutical Manufacturing. Org Process Res Dev 2023, 27, 409-422. https://doi.org/https://doi.org/10.1021/acs.oprd.2c00314.
(27) Jan-Hendrik Schöbel; Frederik Winkelmann; Joel Bicker; Michael Felderhoff. Mechanochemical Kilogram-Scale Synthesis of Rac-lbuprofen:Nicotinamide Co-Crystals Using a Drum Mill. RSC Mechanochemistry 2025, 2, 224-229. https://doi.org/10.1039/D4MR00096J.
(28) Germann, L. S.; Arhangelskis, M.; Etter, M.; Dinnebier, R. E.; Friščić, T. Challenging the Ostwald Rule of Stages in Mechanochemical Cocrystallisation. Chem Sci 2020, 11, 10092-10100. https://doi.org/10.1039/d0sc03629c.
(29) Braga, D.; Giaffreda, S. L.; Grepioni, F.; Pettersen, A.; Maini, L.; Curzi, M.; Polito, M. Mechanochemical Preparation of Molecular and Supramolecular Organometallic Materials and Coordination Networks. Dalton Transactions 2006, 6, 1249-1263. https://doi.org/10.1039/B516165G.
(30) Frisić, T.; Childs, S. L.; Rizvi, S. A. A.; Jones, W. The Role of Solvent in Mechanochemical and Sonochemical Cocrystal Formation: A Solubility-Based Approach for Predicting Cocrystallisation Outcome. CrystEngComm 2009, 11, 418-426. https://doi.org/10.1039/B815174A.
(31) Crawford, D. E.; Casaban, J.; Crawford, D. E.; Casaban, J. Recent Developments in Mechanochemical Materials Synthesis by Extrusion. Advanced Materials 2016, 28, 5747-5754. https://doi.org/10.1002/ADMA.201505352.
(32) Lennox, C. B.; Borchers, T. H.; Gonnet, L.; Barrett, C. J.; Koenig, S. G.; Nagapudi, K.; Friščić, T. Direct Mechanocatalysis by Resonant Acoustic Mixing (RAM). Chem Sci 2023, 14, 7475-7481. https://doi.org/10.1039/D3SCO1591B.
(33) Michalchuk, A. A. L.; Hope, K. S.; Kennedy, S. R.; Blanco, M. V.; Boldyreva, E. V.; Pulham, C. R. Ball-Free Mechanochemistry: In Situ Real-Time Monitoring of Pharmaceutical Co-Crystal Formation by Resonant Acoustic Mixing. Chemical Communications 2018, 54, 4033-4036. https://doi.org/10.1039/C8CC02187B.
(34) Teoh, Y.; Ayoub, G.; Huskić, I.; Titi, H. M.; Nickels, C. W.; Herrmann, B.; FriščićT.
SpeedMixing: Rapid Tribochemical Synthesis and Discovery of Pharmaceutical Cocrystals without Milling or Grinding Media**. Angewandte Chemie International Edition 2022, 61. https://doi.org/10.1002/anie.202206293.
(35) Anastas, P.; Eghbali, N. Green Chemistry: Principles and Practice. Chem. Soc. Rev. 2010, 39, 301-312. https://doi.org/10.1039/B918763B.
(36) Ardila-Fierro, K. J.; Hernández, J. G. Sustainability Assessment of Mechanochemistry by Using the Twelve Principles of Green Chemistry. Chem Sus Chem 2021, 14, 2145-2162. https://doi.org/10.1002/CSSC.202100478.
(37) Geppi, M.; Mollica, G.; Borsacchi, S.; Veracini, C. A. Solid-State NMR Studies of Pharmaceutical Systems. Appl Spectrosc Rev 2008, 43, 202-302. https://doi.org/10.1080/05704920801944338.
(38) Harris, R. K. NMR Studies of Organic Polymorphs & Solvates. Analyst 2006, 131, 351-373. https://doi.org/10.1039/B516057J.
(39) Pindelska, E.; Sokal, A.; Kolodziejski, W. Pharmaceutical Cocrystals, Salts and Polymorphs: Advanced Characterization Techniques. Adv Drug Deliv Rev 2017, 117, 111-146. https://doi.org/10.1016/J.ADDR.2017.09.014.
(40) Stephenson, G. A.; Forbes, R. A.; Reutzel-Edens, S. M. Characterization of the Solid State: Quantitative Issues. Adv Drug Deliv Rev 2001, 48, 67-90. https://doi.org/10.1016/S0169-409X(01)00099-0.
(41) Vogt, F. G. Solid-State NMR in Drug Discovery and Development. In New Applications of NMR in Drug Discovery and Development; 2013; pp 43-100. https://doi.org/10.1039/9781849737661-00043.
(42) Brittain, H. G. Spectroscopy of Pharmaceutical Solids; CRC Press, 2006. https://doi.org/10.1201/9780849361333.
(43) Shankland, K. An Overview of Powder X-Ray Diffraction and Its Relevance to Pharmaceutical Crystal Structures. 2016, 293-314. https://doi.org/10.1007/978-1-4939-4029-5_8.
(44) Brittain, H. G.; Bogdanowich, S. J.; Bugay, D. E.; DeVincentis, J.; Lewen, G.; Newman, A. W. Physical Characterization of Pharmaceutical Solids. Pharm Res 1991, 8, 963-973. https://doi.org/10.1023/A:1015888520352/.
(45) Bugay, D. E. Characterization of the Solid-State: Spectroscopic Techniques. Adv Drug Deliv Rev 2001, 48, 43-65. https://doi.org/10.1016/S0169-409X(01)₀₀₁₀₁-6.
(46) Namespetra, A. M.; Hirsh, D. A.; Hildebrand, M. P.; Sandre, A. R.; Hamaed, H.; Rawson, J. M.; Schurko, R. W. 35Cl Solid-State NMR Spectroscopy of HCl Pharmaceuticals and Their Polymorphs in Bulk and Dosage Forms. CrystEngComm 2016, 18, 6213-6232. https://doi.org/10.1039/c6ce01069e.
(47) Bryce, D. L.; Sward, G. D. Chlorine-35/37 NMR Spectroscopy of Solid Amino Acid Hydrochlorides: Refinement of Hydrogen-Bonded Proton Positions Using Experiment and Theory. J Phys Chem B 2006, 110, 26461-26470. https://doi.org/10.1021/jp065878c.
(48) Szell, P. M. J.; Bryce, D. L. 35Cl Solid-State NMR and Computational Study of Chlorine Halogen Bond Donors in Single-Component Crystalline Chloronitriles. J. Phys. Chem. C 2016, 120, 11121-11130. https://doi.org/10.1021/acs.jpcc.6b02806.
(49) Struppe, J.; Quinn, C. M.; Sarkar, S.; Gronenborn, A. M.; Polenova, T. Ultrafast 1 H MAS NMR Crystallography for Natural Abundance Pharmaceutical Compounds. Mol Pharm 2020, 17, 674-682. https://doi.org/10.1021/acs.molpharmaceut.9b01157.
(50) Vogt, F. G.; Williams, G. R.; Strohmeier, M.; Johnson, M. N.; Copley, R. C. B. Solid-State NMR Analysis of a Complex Crystalline Phase of Ronacaleret Hydrochloride. Journal of Physical Chemistry B 2014, 118, 10266-10284. https://doi.org/https://doi.org/10.1021/jp505061j.
(51) Pandey, M. K.; Kato, H.; Ishii, Y.; Nishiyama, Y. Two-Dimensional Proton-Detected 35C1/1H Correlation Solid-State NMR Experiment under Fast Magic Angle Sample Spinning: Application to Pharmaceutical Compounds. Physical Chemistry Chemical Physics 2016, 18, 6209-6216. https://doi.org/10.1039/C5CP06042G.
(52) Stirk, A. J.; Holmes, S. T.; Souza, F. E. S.; Hung, I.; Gan, Z.; Britten, J. F.; Rey, A. W.; Schurko, R. W. An Unusual Ionic Cocrystal of Ponatinib Hydrochloride: Characterization by Single-Crystal X-Ray Diffraction and Ultra-High Field NMR Spectroscopy. CrystEngComm 2024, 26, 1219-1233. https://doi.org/10.1039/D3CE01062G.
(53) Hildebrand, M.; Hamaed, H.; Namespetra, A. M.; Donohue, J. M.; Fu, R.; Hung, I.; Gan, Z.; Schurko, R. W. 35 Cl Solid-State NMR of HCl Salts of Active Pharmaceutical Ingredients: Structural Prediction, Spectral Fingerprinting and Polymorph Recognition. CrystEngComm 2014, 16, 7334-7356. https://doi.org/10.1039/C4CE00544A.
(54) Abdulla, L. M.; Peach, A. A.; Holmes, S. T.; Dowdell, Z. T.; Watanabe, L. K.; Iacobelli, E. M.; Hirsh, D. A.; Rawson, J. M.; Schurko, R. W. Synthesis and Characterization of Xylazine Hydrochloride Polymorphs, Hydrates, and Cocrystals: A 35Cl Solid-State NMR and DFT Study. Cryst Growth Des 2023, 23, 3412-3426. https://doi.org/10.1021/acs.cgd.2c01539.
(55) Hirsh, D. A.; Holmes, S. T.; Chakravarty, P.; Peach, A. A.; Dipasquale, A. G.; Nagapudi, K.; Schurko, R. W. In Situ Characterization of Waters of Hydration in a Variable-Hydrate Active Pharmaceutical Ingredient Using 35Cl Solid-State NMR and X-Ray Diffraction. Cryst Growth Des 2019, 19, 7349-7362. https://doi.org/10.1021/acs.cgd.9b01218.
(56) Holmes, S. T.; Hook, J. M.; Schurko, R. W. Nutraceuticals in Bulk and Dosage Forms: Analysis by 35Cl and 14N Solid-State NMR and DFT Calculations. Mol Pharm 2022, 19, 440-455. https://doi.org/10.1021/ACS.MOLPHARMACEUT.1C00708.
(57) Peach, A. A.; Hirsh, D. A.; Holmes, S. T.; Schurko, R. W. Mechanochemical Syntheses and 35 Cl Solid-State NMR Characterization of Fluoxetine HCl Cocrystals. CrystEngComm 2018, 20, 2780-2792. https://doi.org/10.1039/C8CE00378E.
(58) Dowdell, Z. T.; Peach, A. A.; Purdie, J. P.; Holmes, S. T.; Watanabe, L. K.; Rawson, J. M.; Schurko, R. W. Mechanochemical Synthesis and 35 Cl NMR Crystallography of Ionic Cocrystals of Phenothiazine Drugs. CrystEngComm 2025, 27, 3752-3768. https://doi.org/10.1039/D5CE00426H.
(59) Vojvodin, C. S.; Sanchez, J. E.; Holmes, S. T.; Lin, X.; Schurko, R. W. Mechanochemical Synthesis and NMR Crystallography of Multicomponent Crystals: New Perspectives from Chlorine and Alkali Metal Solid-State NMR Spectroscopy. Cryst Growth Des 2025, 25, 9591-9609. https://doi.org/10.1021/ACS.CGD.5C00540.
(60) Vojvodin, C. S.; Holmes, S. T.; Watanabe, L. K.; Rawson, J. M.; Schurko, R. W. Multi-Component Crystals Containing Urea: Mechanochemical Synthesis and Characterization by 35 Cl Solid-State NMR Spectroscopy and DFT Calculations. CrystEngComm 2022, 24, 2626-2641. https://doi.org/10.1039/DiCE01610E.
(61) A. Peach, A.; H. Fleischer, C.; Kirill Levin; T. Holmes, S.; E. Sanchez, J.; W. Schurko, R. Quadrupolar NMR Crystallography Guided Crystal Structure Prediction (QNMRX-CSP). CrystEngComm 2024, 26, 4782-4803. https://doi.org/i0.1039/D3CE01306E.
(62) H. Fleischer, C.; T. Holmes, S.; Kirill Levin; L. Veinberg, S.; W. Schurko, R. Characterization of Ephedrine HCl and Pseudoephedrine HCl Using Quadrupolar NMR Crystallography Guided Crystal Structure Prediction. Faraday Discuss 2025, 255, 88-118. https://doi.org/10.1039/D4FD00089G.
(63) Bryce, D. L.; Sward, G. D. Solid-State NMR Spectroscopy of the Quadrupolar Halogens: Chlorine-35/37, Bromine-79/81, and Iodine-127. Magnetic Resonance in Chemistry 2006, 44, 409-450. https://doi.org/10.1002/MRC.1741.
(64) Perras, F. A.; Bryce, D. L.; Perras, F. A.; Bryce, D. L. Direct Investigation of Covalently Bound Chlorine in Organic Compounds by Solid-State 35Cl NMR Spectroscopy and Exact Spectral Line-Shape Simulations. Angewandte Chemie International Edition 2012, 51, 4227-4230. https://doi.org/10.1002/ANIE.201200728.
(65) Bryce, D. L.; Sward, G. D. Chlorine-35/37 NMR Spectroscopy of Solid Amino Acid Hydrochlorides: Refinement of Hydrogen-Bonded Proton Positions Using Experiment and Theory. J Phys Chem B 2006, 110, 26461-26470. https://doi.org/10.1021/jp065878c.
(66) Bryce, D. L.; Gee, M.; Wasylishen, R. E. High-Field Chlorine NMR Spectroscopy of Solid Organic Hydrochloride Salts: A Sensitive Probe of Hydrogen Bonding Environment. J Phys Chem A 2001, 105, 10413-10421. https://doi.org/10.1021/jp011962a.
(67) Chapman, R. P.; Widdifield, C. M.; Bryce, D. L. Solid-State NMR of Quadrupolar Halogen Nuclei. Prog Nucl Magn Reson Spectrosc 2009, 55, 215-237. https://doi.org/10.1016/J.PNMRS.2009.05.001.
(68) Chapman, R. P.; Hiscock, J. R.; Gale, P. A.; Bryce, D. L. A Solid-State 35/37Cl NMR Study of a Chloride Ion Receptor and a GIPAW-DFT Study of Chlorine NMR Interaction Tensors in Organic Hydrochlorides. Can J Chem 2011, 89, 822-834. https://doi.org/doi.org/10.1139/v10-177.
(69) Bryce, D. L.; Sward, G. D.; Adiga, S. Solid-State 35/37Cl NMR Spectroscopy of Hydrochloride Salts of Amino Acids Implicated in Chloride Ion Transport Channel Selectivity: Opportunities at 900 MHz. J Am Chem Soc 2006, 128, 2121-2134. https://doi.org/doi.org/10.1021/ja057253i.
(70) Perumalla, S. R.; Sun, C. C. Design and Synthesis of Solid State Structures with Conjugate Acid-Base Pair Interactions. CrystEngComm 2012, 14, 3851-3853. https://doi.org/10.1039/C2CE25193K/.
(71) Takusagawa, F.; Shimada, A. Isonicotinic Acid. Acta Cryst. 1976, B32, 1925-1927. https://doi.org/10.1107/S0567740876006754.
(72) Wright, W. B.; King, G. S. D. The Crystal Structure of Nicotinic Acid. Acta Cryst. 1953, 6, 305-317. https://doi.org/10.1107/S0365110X53000867.
(73) Miwa, Y.; Mizuno, T.; Tsuchida, K.; Taga, T.; Iwata, Y. Experimental Charge Density and Electrostatic Potential in Nicotinamide. Acta Cryst. 1999, B55, 78-84. https://doi.org/10.1107/SO108768198007848.
(74) Aakeröy, C. B.; Beatty, A. M.; Helfrich, B. A.; Nieuwenhuyzen, M. Do Polymorphic Compounds Make Good Cocrystallizing Agents?A Structural Case Study That Demonstrates the Importance of Synthon Flexibility. Cryst Growth Des 2003, 3, 159-165. https://doi.org/doi.org/10.1021/cg025593z.
(75) Tyler, A. R.; Ragbirsingh, R.; McMonagle, C. J.; Waddell, P. G.; Heaps, S. E.; Steed, J. W.; Thaw, P.; Hall, M. J.; Probert, M. R. Encapsulated Nanodroplet Crystallization of Organic-Soluble Small Molecules. Chem 2020, 6, 1755-1765. https://doi.org/10.1016/J.CHEMPR.2020.04.009.
(76) Anitha, R.; Gunasekaran, M.; Kumar, S. S.; Athimoolam, S.; Sridhar, B. Single Crystal XRD, Vibrational and Quantum Chemical Calculation of Pharmaceutical Drug Paracetamol: A New Synthesis Form. Spectrochim Acta A Mol Biomol Spectrosc 2015, 150, 488-498. https://doi.org/10.1016/J.SAA.2015.05.091.
(77) Takusagawa, F.; Shimada, A. The Crystal Structure of Picolinic Acid. Chem Lett 1973, 2, 1089-1090. https://doi.org/10.1246/CL.1973.1089.
(78) Takano, T.; Sasada, Y.; Kakudo, M. The Crystal and Molecular Structure of Picolinamide. Acta Cryst. 1966, 21, 514-522. https://doi.org/10.1107/S0365110X66003396.
(79) David, W. I. F.; Shankland, K. Routine Determination of Molecular Crystal Structures from Powder Diffraction Data. Chemical Communications 1998, 8, 931-932. https://doi.org/10.1039/A800855H.
(80) CrystalDiffract Overview http://crystalmaker.com/crystaldiffract/(accessed 2022-11-11).
(81) Sheldrick, G. M.; IUCr. SHELXT—Integrated Space-Group and Crystal-Structure Determination. Acta Cryst. 2015, 71, 3-8. https://doi.org/10.1107/S2053273314026370.
(82) Sheldrick, G. M. Crystal Structure Refinement with SHELXL. Acta Cryst 2015, 71, 3-8. https://doi.org/10.1107/S2053229614024218.
(83) Hahn, E. L. Spin Echoes. Physical Review 1950, 80, 580. https://doi.org/10.1103/PhysRev.80.580.
(84) Larsen, F. H.; Jakobsen, H. J.; Ellis, P. D.; Nielsen, N. C. QCPMG-MAS NMR of Half-Integer Quadrupolar Nuclei. Journal of Magnetic Resonance 1998, 131, 144-147. https://doi.org/10.1006/JMRE.1997.1341.
(85) Larsen, F. H.; Jakobsen, H. J.; Ellis, P. D.; Nielsen, N. C. Sensitivity-Enhanced Quadrupolar-Echo NMR of Half-Integer Quadrupolar Nuclei. Magnitudes and Relative Orientation of Chemical Shielding and Quadrupolar Coupling Tensors. Journal of Physical Chemistry A 1997, 101, 8597-8606. https://doi.org/10.1021/jp971547b.
(86) Hung, I.; Gan, Z. On the Practical Aspects of Recording Wideline QCPMG NMR Spectra. Journal of Magnetic Resonance 2010, 204, 256-265. https://doi.org/10.1016/J.JMR.2010.03.001.
(87) Lipton, A. S.; Sears, J. A.; Ellis, P. D. A General Strategy for the NMR Observation of Half-Integer Quadrupolar Nuclei in Dilute Environments. Journal of Magnetic Resonance 2001, 151, 48-59. https://doi.org/10.1006/JMRE.2001.2353.
(88) Siegel, R.; Nakashima, T. T.; Wasylishen, R. E. Application of Multiple-Pulse Experiments to Characterize Broad NMR Chemical-Shift Powder Patterns from Spin-1/2 Nuclei in the Solid State. J. Phys. Chem. B 2004, 108, 2218-2226. https://doi.org/10.1021/jp031048c.
(89) Hung, I.; Rossini, A. J.; Schurko, R. W. Application of the Carr-Purcell Meiboom-Gill Pulse Sequence for the Acquisition of Solid-State NMR Spectra of Spin-1/2 Nuclei. J. Phys. Chem. A 2004, 108, 7112-7120. https://doi.org/doi.org/10.1021/jp040l123.
(90) O'Dell, L. A. The WURST Kind of Pulses in Solid-State NMR. Solid State Nucl Magn Reson 2013, 55-56, 28-41. https://doi.org/10.1016/J.SSNMR.2013.10.003.
(91) O'Dell, L. A.; Schurko, R. W. QCPMG Using Adiabatic Pulses for Faster Acquisition of Ultra-Wideline NMR Spectra. Chem Phys Lett 2008, 464, 97-102. https://doi.org/10.1016/J.CPLETT.2008.08.095.
(92) O'Dell, L. A.; Rossini, A. J.; Schurko, R. W.; O'Dell, L. A.; Rossini, A. J.; Schurko, R. W. Acquisition of Ultra-Wideline NMR Spectra from Quadrupolar Nuclei by Frequency Stepped WURST-QCPMG. Chem Phys Lett 2009, 468, 330-335.
(93) Larsen, F. H.; Jakobsen, H. J.; Ellis, P. D.; Nielsen, N. C. QCPMG-MAS NMR of Half-Integer Quadrupolar Nuclei. Journal of Magnetic Resonance 1998, 131, 144-147. https://doi.org/10.1006/JMRE.1997.1341.
(94) Bloch, F. Nuclear Induction. Physical Review 1946, 70, 460-474. https://doi.org/10.1103/PHYSREV.70.460.
(95) Comellas, G.; Lopez, J. J.; Nieuwkoop, A. J.; Lemkau, L. R.; Rienstra, C. M.
Straightforward, Effective Calibration of SPINAL-64 Decoupling Results in the Enhancement of Sensitivity and Resolution of Biomolecular Solid-State NMR. Journal of Magnetic Resonance 2011, 209, 131-135. https://doi.org/10.1016/J.JMR.2010.12.011.
(96) van Meerten, S. G. J.; Franssen, W. M. J.; Kentgens, A. P. M. SsNake: A Cross-Platform Open-Source NMR Data Processing and Fitting Application. Journal of Magnetic Resonance 2019, 301, 56-66. https://doi.org/10.1016/J.JMR.2019.02.006.
(97) Eichele, K. WSolids1 ver. 1.21.7 http://anorganik.uni-tuebingen.de/klaus/soft/index.php?p=wsolids1/wsolids1.
(98) Perras, F. A.; Paterson, A. L. Automatic Fitting of Multiple-Field Solid-State NMR Spectra. Solid State Nucl Magn Reson 2024, 131, 101935. https://doi.org/10.1016/J.SSNMR.2024.101935.
(99) David, W. I. F.; Shankland, K.; Shankland, N. Routine Determination of Molecular Crystal Structures from Powder Diffraction Data. Chemical Communications 1998, No. 8, 931-932. https://doi.org/10.1039/A800855H.
(100) Holmes, S. T.; Schurko, R. W. Refining Crystal Structures with Quadrupolar NMR and Dispersion-Corrected Density Functional Theory. J. Phys. Chem. C 2018, 122, 1809-1820. https://doi.org/10.1021/acs.jpcc.7b12314.
(101) Holmes, S. T.; Vojvodin, C. S.; Schurko, R. W. Dispersion-Corrected DFT Methods for Applications in Nuclear Magnetic Resonance Crystallography. J. Phys. Chem. A 2020, 124, 10312-10323. https://doi.org/10.1021/acs.jpca.0c06372.
(102) Payne, M. C.; Teter, M. P.; Allan, D. C.; Arias, T. A.; Joannopoulos, J. D. Iterative Minimization Techniques for Ab Initio Total-Energy Calculations: Molecular Dynamics and Conjugate Gradients. Rev Mod Phys 1992, 64, 1045. https://doi.org/10.1103/RevModPhys.64.1045.
(103) Clark, S. J.; Segall, M. D.; Pickard, C. J.; Hasnip, P. J.; Probert, M. I. J.; Refson, K.; Payne, M. C. First Principles Methods Using CASTEP. Z Kristallogr Cryst Mater 2005, 220, 567-570. https://doi.org/10.1524/zkri.220.5.567.65075.
(104) Hammer, B.; Hansen, L. B.; Norskov, J. K. Improved Adsorption Energetics within Density-Functional Theory Using Revised Perdew-Burke-Ernzerhof Functionals. Phys Rev B 1999, 59, 7413. https://doi.org/10.1103/PhysRevB.59.7413.
(105) Yates, J. R.; Pickard, C. J.; Mauri, F. Calculation of NMR Chemical Shifts for Extended Systems Using Ultrasoft Pseudopotentials. Phys Rev B 2007, 76, 024401. https://doi.org/10.1103/PhysRevB.76.024401.
(106) Pfrommer, B. G.; Côté, M.; Louie, S. G.; Cohen, M. L. Relaxation of Crystals with the Quasi-Newton Method. J Comput Phys 1997, 131, 233-240. https://doi.org/10.1006/JCPH.1996.5612.
(107) Grimme, S. Semiempirical GGA-Type Density Functional Constructed with a Long-Range Dispersion Correction. J Comput Chem 2006, 27, 1787-1799. https://doi.org/10.1002/jcc.20495.
(108) McNellis, E. R.; Meyer, J.; Reuter, K. Azobenzene at Coinage Metal Surfaces: Role of Dispersive van Der Waals Interactions. Phys Rev B Condens Matter Mater Phys 2009, 80, 205414. https://doi.org/10.1103/PHYSREVB.80.205414.
(109) Holmes, S. T.; luliucci, R. J.; Mueller, K. T.; Dybowski, C. Semi-Empirical Refinements of Crystal Structures Using 170 Quadrupolar-Coupling Tensors. J Chem Phys 2017, 146, 064201. https://doi.org/10.1063/1.4975170.
(110) Pickard, C. J.; Mauri, F. All-Electron Magnetic Response with Pseudopotentials: NMR Chemical Shifts. Phys Rev B 2001, 63, 245101. https://doi.org/10.1103/PhysRevB.63.245101.
(111) Adiga, S.; Aebi, D.; Bryce, D. L. EFGShield—A Program for Parsing and Summarizing the Results of Electric Field Gradient and Nuclear Magnetic Shielding Tensor Calculations. Can J Chem 2007, 85, 496-505. https://doi.org/10.1139/v07-069.
(112) Tumanova, N.; Tumanov, N.; Robeyns, K.; Fischer, F.; Fusaro, L.; Morelle, F.; Ban, V.; Hautier, G.; Filinchuk, Y.; Wouters, J.; Leyssens, T.; Emmerling, F. Opening Pandora's Box: Chirality, Polymorphism, and Stoichiometric Diversity in Flurbiprofen/Proline Cocrystals. Cryst Growth Des 2018, 18, 954-961. https://doi.org/doi.org/10.1021/acs.cgd.7b01436.
(113) Tumanova, N.; Tumanov, N.; Fischer, F.; Morelle, F.; Ban, V.; Robeyns, K.; Filinchuk, Y.; Wouters, J.; Emmerling, F.; Leyssens, T. Exploring Polymorphism and Stoichiometric Diversity in Naproxen/Proline Cocrystals. CrystEngComm 2018, 20, 7308-7321. https://doi.org/10.1039/C8CE01338A.
(114) Hasa, D.; Schneider Rauber, G.; Voinovich, D.; Jones, W. Cocrystal Formation through Mechanochemistry: From Neat and Liquid-Assisted Grinding to Polymer-Assisted Grinding. Angewandte Chemie 2015, 127, 7479-7483. https://doi.org/10.1002/ANGE.201501638.

(115) Lukin, S.; Germann, L. S.; Fri§ cid, T.; Halasz, I. Toward Mechanistic Understanding of Mechanochemical Reactions Using Real-Time in Situ Monitoring. Acc Chem Res 2022, 55, 1262-1277. https://doi.org/i0.1021/acs.accounts.2c00062.
(116) Gogoi, D.; Kalita, K. J.; Biswakarma, N.; Arhangelskis, M.; Deka, R. C.; Thakuria, R. Exploring Polymorphism, Stoichiometric Diversity and Simultaneous Existence of Salt and Cocrystal during Cocrystallization Using Mechanochemistry. RSC Mechanochemistry 2024, 1, 452-464. https://doi.org/10.1039/D3MR00022B.
(117) Hasa, D.; Carlino, E.; Jones, W. Polymer-Assisted Grinding, a Versatile Method for Polymorph Control of Cocrystallization. 2016, 17, 674-682. https://doi.org/10.1021/acs.cgd.6b00084.
(118) Ma, L.; Zheng, Q.; Unruh, D. K.; Hutchins, K. M. Reversible Interconversion of Pharmaceutical Salt Polymorphs Facilitated by Mechanical Methods. Chemical Communications 2023, 59, 7779-7782. https://doi.org/10.1039/D3CC02188B.
(119) Schmidt, R.; Martin Scholze, H.; Stolle, A. Temperature Progression in a Mixer Ball Mill. International Journal of Industrial Chemistry 2016, 7, 181-186. https://doi.org/https://doi.org/10.1007/s40090-016-0078-8.
(120) Gurau, G.; Kelley, S. P.; Di Bona, K. R.; Smiglak, M.; Rogers, R. D. Anhydrous Caffeine Hydrochloride and Its Hydration. Cryst Growth Des 2012, 12, 4658-4662. https://doi.org/10.1021/cg300878j.
(121) Peach, A. A.; Holmes, S. T.; MacGillivray, L. R.; Schurko, R. W. The Formation and Stability of Fluoxetine HCl Cocrystals Investigated by Multicomponent Milling. CrystEngComm 2023, 25, 213-224. https://doi.org/10.1039/D2CE01341J.
(122) Fischer, F.; Lubjuhn, D.; Greiser, S.; Rademann, K.; Emmerling, F. Supply and Demand in the Ball Mill: Competitive Cocrystal Reactions. Cryst Growth Des 2016, 16, 5843-5851. https://doi.org/10.1021/acs.cgd.6b00928.
(123) Jellinek, H. H. G.; Urwin, J. R. Ultraviolet Absorption Spectra and Dissociation Constants of Picolinic, Isonicotinic Acids and Their Amides. Journal of Physical Chemistry 1954, 58, 548-550. https://doi.org/10.1021/j150517a009.
(124) Day, G. M.; Chisholm, J.; Shan, N.; Motherwell, W. D. S.; Jones, W. An Assessment of Lattice Energy Minimization for the Prediction of Molecular Organic Crystal Structures. Cryst Growth Des 2004, 4, 1327-1340. https://doi.org/10.1021/cg0498148.
(125) Aitipamula, S.; Banerjee, R.; Bansal, A. K.; Biradha, K.; Cheney, M. L.; Choudhury, A. R.; Desiraju, G. R.; Dikundwar, A. G.; Dubey, R.; Duggirala, N.; Ghogale, P. P.; Ghosh, S.; Goswami, P. K.; Goud, N. R.; Jetti, R. R. K. R.; Karpinski, P.; Kaushik, P.; Kumar, D.; Kumar, V.; Moulton, B.; Mukherjee, A.; Mukherjee, G.; Myerson, A. S.; Puri, V.; Ramanan, A.; Rajamannar, T.; Reddy, C. M.; Rodriguez-Hornedo, N.; Rogers, R. D.; Row, T. N. G.; Sanphui, P.; Shan, N.; Shete, G.; Singh, A.; Sun, C. C.; Swift, J. A.; Thaimattam, R.; Thakur, T. S.; Kumar Thaper, R.; Thomas, S. P.; Tothadi, S.; Vangala, V. R.; Variankaval, N.; Vishweshwar, P.; Weyna, D. R.; Zaworotko, M. J. Polymorphs, Salts, and Cocrystals: What's in a Name?Cryst Growth Des 2012, 12, 2147-2152. https://doi.org/10.1021/cg3002948.
(126) Taylor, C. R.; Day, G. M. Evaluating the Energetic Driving Force for Cocrystal Formation. Cryst Growth Des 2018, 18, 892-904. https://doi.org/10.1021/acs.cgd.7b01375.
(127) Stone, J.; Edgar, J. O.; Rutherford, J.; Gill-Olivas, B.; Tranter, M.; Gould, J. A.; Xavier, C. M.; Telling, J.; Li, Y.; Wu, X.; Jiang, H. Flash Heating Boosts the Potential for Mechanochemical Energy Sources for Subglacial Ecosystems. Frontiers in Geochemistry 2023, 1, 1180893. https://doi.org/10.3389/FGEOC.2023.1180893.
(128) Michalchuk, A. A. L. On the Physical Processes of Mechanochemically Induced Transformations in Molecular Solids. Chemical Communications 2024, 60, 14750-14761. https://doi.org/10.1039/D4CC04062G.
(129) Ganduri, R.; Cherukuvada, S.; Sarkar, S.; Guru Row, T. N. Manifestation of Cocrystals and Eutectics among Structurally Related Molecules: Towards Understanding the Factors That Control Their Formation. CrystEngComm 2017, 19, 1123-1132. https://doi.org/10.1039/C6CE02003H.
(130) Beyer, M. K.; Clausen-Schaumann, H. Mechanochemistry: The Mechanical Activation of Covalent Bonds. Chem Rev 2005, 105, 2921-2948. https://doi.org/10.1021/cr030697h.
(131) Boldyreva, E. Mechanochemistry of Inorganic and Organic Systems: What Is Similar, What Is Different?Chem Soc Rev 2013, 42, 7719-7738. https://doi.org/10.1039/C3CS60052A.
(132) James, S. L.; Adams, C. J.; Bolm, C.; Braga, D.; Collier, P.; Frišcic, T.; Grepioni, F.; Harris, K. D. M.; Hyett, G.; Jones, W.; Krebs, A.; Mack, J.; Maini, L.; Orpen, A. G.; Parkin, I. P.; Shearouse, W. C.; Steed, J. W.; Waddell, D. C. Mechanochemistry: Opportunities for New and Cleaner Synthesis. Chem Soc Rev 2011, 41, 413-447. https://doi.org/10.1039/ClCS15171A.
(133) Gui, Y. Solid Form Screenings in Pharmaceutical Development: A Perspective on Current Practices. Pharm Res 2023, 40, 2347-2354. https://doi.org/i0.1007/s11095-023-03573-2.
(134) Aaltonen, J.; Allesø, M.; Mirza, S.; Koradia, V.; Gordon, K. C.; Rantanen, J. Solid Form Screening—A Review. European Journal of Pharmaceutics and Biopharmaceutics 2009, 71, 23-37. https://doi.org/10.1016/J.EJPB.2008.07.014.
(135) Gowri, S.; Uma Devi, T.; Sajan, D.; Surendra Dilip, C.; Chandramohan, A.; Lawrence, N. Crystal Growth, Spectral, Optical and Thermal Properties of Semiorganic Nonlinear Optical Material: Picolinic Acid Hydrochloride. Spectrochim Acta A Mol Biomol Spectrosc 2013, 110, 28-35. https://doi.org/10.1016/J.SAA.2013.03.007.
(136) Bryce, D. L. Tensor Interplay. eMagRes 2008. https://doi.org/10.1002/9780470034590.EMRSTM1039.
(137) Lacková, D.; Ondrejkovičová, I.; Padělková, Z.; Koman, M. Syntheses, Crystal Structures, and IR Spectra of Isonicotinamide-Isonicotinamidium Bis(lsonicotinamide)-Tetrakis(Isothiocyanato)Ferrate(III) and Isonicotinamidium Chloride. J Coord Chem 2014, 67, 1652-1663. https://doi.org/10.1080/00958972.2014.917634.
(138) Holmes, S. T.; Iuliucci, R. J.; Mueller, K. T.; Dybowski, C. Semi-Empirical Refinements of Crystal Structures Using 170 Quadrupolar-Coupling Tensors. J Chem Phys 2017, 146, 064201. https://doi.org/10.1063/1.4975170.
(139) Desiraju, G. R. Crystal Engineering: A Holistic View. Angewandte Chemie International Edition 2007, 46, 8342-8356. https://doi.org/i0.1002/ANIE.200700534.
(140) Hirsh, D. A.; Holmes, S. T.; Chakravarty, P.; Peach, A. A.; DiPasquale, A. G.; Nagapudi, K.; Schurko, R. W. In Situ Characterization of Waters of Hydration in a Variable-Hydrate Active Pharmaceutical Ingredient Using 35 Cl Solid-State NMR and X-Ray Diffraction. Cryst Growth Des 2019, 19, 7349-7362. https://doi.org/10.1021/acs.cgd.9b01218.
(141) Hirsh, D. A.; Su, Y.; Nie, H.; Xu, W.; Stueber, D.; Variankaval, N.; Schurko, R. W. Quantifying Disproportionation in Pharmaceutical Formulations with 35 Cl Solid-State NMR. Mol Pharm 2018, 15, 4038-4048. https://doi.org/10.1021/acs.molpharmaceut.8b00470.

1. A method for producing a salt of a solid organic compound, the method comprising:
combining in the solid state the solid organic compound comprising one or more protonatable groups and an acid composition, wherein the amount of acid in the acid composition is about stoichiometric or less than stoichiometric relative to the one or more protonatable groups of the solid organic compound to produce a precursor mixture; and
mixing the precursor mixture by mechanochemical milling to produce the salt of the solid organic compound.

2. The method of claim 1, wherein the acid comprises H₂SO₄, HCl, HBr, HI, HClO₄, HNO₃, H₃PO₄, H₃PO₃, HF, HCN, H₂CrO₄, or any combination thereof.

3. The method of claim 1, wherein the acid composition is from 0.1 μL per mg of the solid organic compound to about 10 μL per mg.

4. The method of claim 1, wherein the acid composition comprises a polar solvent.

5. The method of claim 4, wherein the polar solvent comprises water, an alcohol, or a combination thereof.

6. The method of claim 1, wherein the acid composition comprises HCl and a polar solvent comprising methanol, ethanol, water, or any combination thereof.

7. The method of claim 1, wherein the acid composition has a concentration of 0.5 M to less than 15 M.

8. The method of claim 1, wherein a ratio of a weight of the solid organic compound to a volume of the acid composition is from about 1:1 to about 4:1.

9. The method of claim 1, wherein the mechanochemical milling comprises ball milling, resonant acoustic mixing, speed mixing, extrusion, planetary milling, or grinding via mortar and pestle or an automated mill.

10. The method of claim 1, wherein the precursor mixture is mixed by mechanochemical milling for about 1 minute to about 30 minutes at a temperature less than or equal to 25° C., and at a frequency of about 10 Hz to about 100 Hz.

11. The method of claim 1, wherein the protonatable group comprises a nitrogen bearing group.

12. The method of claim 1, wherein the protonatable group comprises an amino group, a nitrogen-bearing heteroaryl group, a secondary amido group, a hydrazine group, or a guanidinyl group.

13. The method of claim 1, wherein the solid organic compound comprises a pharmaceutical, a nutraceutical, an agrochemical, or an explosive.

14. The method of claim 1, wherein the solid organic compound comprises an amino acid, a peptide, a protein, a nucleic acid, or a small molecule.

15. The method of claim 1, wherein the solid organic compound comprises nicotinamide, isonicotinamide, picolinamide, nicotinic acid, isonicotinic acid, picolinic acid, caffeine, theophylline, theobromine, acetaminophen, creatine, metformin, L-lysine, L-histidine, L-alanine, sulfanilamide, isoniazid, amoxicillin, nicorandil, amlodipine, sulfonamide, or pyrazinamide.

16. The method of claim 1, wherein the acid composition comprises two or more different acids.

17. The method of claim 1, wherein the amount of acid in the acid composition is about stoichiometric relative to each protonatable group of the solid organic compound.

18. The method of claim 1, wherein when the solid organic compound possesses two or more protonatable groups, the amount of acid in the acid composition is about stoichiometric relative to a single protonatable group of the solid organic compound.

19. The method of claim 1, wherein the salt of the solid organic compound is an anhydrous compound, a monohydrate compound, or a combination thereof.

20. The method of claim 1, wherein the salt of the solid organic compound is isonicotinamide-isonicotinamidium chloride monohydrate (Iso-IsoH:H₂O), picolinamide HCl monohydrate (PicH:H₂O), picolinamide HCl (PicH), picolinamide-picolinamidium chloride (PicH-Pic), picolinic acid hemihydrochloride (PicAH-PicA), and (bis)acetaminophen HCl monohydrate (Acet-AcetH:H₂O).
